(19) United States
(12) Patent Application Publication
(10) Pub. No.: US 2026/0027174 A1
(43) Pub. Date: Jan. 29, 2026

(54) GRAPE SEED EXTRACTS AND RELATED METHODS OF USE

(71) Applicant: E. & J. Gallo Winery, Modesto, CA (US)

(72) Inventors: Mark A. Kelm, Fresno, CA (US); Peter Park, Fresno, CA (US)

(21) Appl. No.: 19/342,163

(22) Filed: Sep. 26, 2025

Related U.S. Application Data
(63) Continuation-in-part of application No. 18/190,682, filed on Mar. 27, 2023.

(51) Int. Cl. A61K 36/87; A61K 31/192; A61K 31/353; A61P 25/28

(52) U.S. Cl. CPC A61K 36/87; A61K 31/192; A61K 31/353; A61P 25/28; A61K 2236/39

(57) ABSTRACT

Grape seed extract compositions and related methods for improving cognition using the same. The grape seed extract compositions may be produced by obtaining a grape seed extract source material; filtering the grape seed extract source material to obtain a first retentate and a first permeate; drying the first retentate to obtain a first grape seed extract having a first mean degree of polymerization; filtering the first permeate to obtain a second retentate and a second permeate; drying the second retentate to obtain a second grape seed extract having a second mean degree of polymerization less than the first mean degree of polymerization; purifying the second permeate to obtain a purified liquid; and drying the purified liquid to obtain a third grape seed extract having a third mean degree of polymerization less than the second mean degree of polymerization; and combining the first, second, or third grape seed extracts.

### CROSS-REFERENCE TO RELATED APPLICATIONS

This application is a continuation-in-part of co-pending U.S. patent application Ser. No. 18/190,682, filed Mar. 27, 2023, the entirety of each of which is herein incorporated by reference.

### TECHNICAL FIELD

The present disclosure relates generally to grape seed extracts and methods for improving cognition using the same.

### BACKGROUND

Grape (Vitis spp.) seed extracts (GSEs) contain chemical compounds known as phenolics. Phenolic compounds can be defined structurally as possessing an aromatic ring with at least one hydroxyl group. Phenolics with multiple aromatic rings and hydroxyl groups can be referred to as polyphenols. Major classes of polyphenols include lignans, stilbenes, and flavonoids (e.g., flavanols). Polymers of flavanol monomers (e.g., catechin and epicatechin) can be referred to as tannins, condensed tannins, or proanthocyanidins (PAs). Sixteen types of proanthocyanidins commonly occur in woody and some herbaceous plants.

The grape berry contains two proanthocyanidin types: procyanidins (catechin, epicatechin, and epicatechin gallate subunits) and prodelphinidins (at least one gallocatechin subunit). Procyanidins can reside in seeds whereas procyanidins and prodelphinidins can both reside in skins. Grape proanthocyanidins can be characterized based on their degree of polymerization (DP), degree of galloylation (DG), subunit composition, branching, and interflavan bond linkage types. There are two types of interflavan bonds, B-type and A-type, joining subunits through C—C and C—O—C bonds, respectively linked. Cyclic proanthocyanidins may also exist. Proanthocyanidins can be present in other plant-based materials, including but not limited to: acacia, açaí, almond, apple, aronia, blueberry, buckwheat, cacao, cranberry, cinnamon, current, grapes, hops, legumes, litchi, oak, pine bark, peanut, persimmon, plum, quebracho, sorghum, tea, and wine.

Proanthocyanidins compounds as well as anthocyanins are capable of traversing the blood-brain barrier and exerting neuroprotective effects through multiple mechanisms, including modulation of inflammatory signaling pathways (e.g., NF-κB, PI3K/Akt, MAPKs), preservation of mitochondrial function, maintenance of calcium homeostasis, and inhibition of apoptosis. These properties collectively contribute to the enhancement of neuronal survival and function. The present disclosure provides novel grape seed extract compositions comprising characteristics not found in other grape seed extracts known in the art. Furthermore, the compositions of the present disclosure may be used to improve cognition of a subject when delivered in an effective amount to said subject.

### SUMMARY OF THE INVENTION

In one aspect, the present disclosure provides a grape seed extract composition for improving cognitive health comprising a first grape seed extract, a second grape seed extract, a third grape seed extract, or a combination thereof. In some embodiments, the first grape seed extract comprises a mean degree of polymerization from about 4.1 to about 5.3, and phenols in an amount from about 61 to about 70 g GAE/100 g, on a dry basis. In other embodiments, the second grape seed extract comprises a mean degree of polymerization from about 2.2 to about 2.7, and phenols in an amount from about 30 to about 60 g GAE/100 g, on a dry basis. In further embodiments, the third grape seed extract comprises a mean degree of polymerization from about 1.2 to about 1.4, and phenols in an amount from about 80 to about 95 g GAE/100 g, on a dry basis. In some embodiments, the composition improves the cognition of a subject when delivered in an effective amount to said subject. In certain embodiments, the composition comprises about 25-75 wt % of the first grape seed extract, about 15-45 wt % of the second grape seed extract, and about 2-20 wt % of the third grape seed extract, wherein a total percentage of the first, second, and third grape seed extracts equals 100%. In other embodiments, the composition comprises: about 25-35 wt % of the first grape seed extract, about 40-45 wt % of the second grape seed extract, and about 20-30 wt % of the third grape seed extract; about 35-45 wt % of the first grape seed extract, about 30-40 wt % of the second grape seed extract, and about 10-20 wt % of the third grape seed extract; about 45-55 wt % of the first grape seed extract, about 25-35 wt % of the second grape seed extract, and about 5-15 wt % of the third grape seed extract; about 55-65 wt % of the first grape seed extract, about 20-30 wt % of the second grape seed extract, and about 2-10 wt % of the third grape seed extract; or about 65-75 wt % of the first grape seed extract, about 15-25 wt % of the second grape seed extract, and about 2-8 wt % of the third grape seed extract. In particular embodiments, the composition comprises about 54 wt % of the first grape seed extract, about 34 wt % of the second grape seed extract, and about 12 wt % of the third grape seed extract. In some embodiments, the first grape seed extract further comprises at least one of: a combination of catechin and epicatechin from about 0% to about 0.5%, by weight, on a dry basis; an epicatechin gallate (ecg) terminal unit percentage from about 0 to about 16; or an epicatechin gallate (ecg) extension unit percentage from about 5.6 to about 15.6. In other embodiments, the second grape seed extract further comprises at least one of: gallic acid in an amount from about 0.3% to about 0.6%, by weight, on a dry basis; a combination of catechin and epicatechin from about 0.3% to about 2.6%, by weight, on a dry basis; an epicatechin gallate (ecg) terminal unit percentage from about 0 to about 14.4; or an epicatechin gallate (ecg) extension unit percentage from about 3.7 to about 11.5. In further embodiments, the third grape seed extract further comprises at least one of: gallic acid in an amount from about 0.4% to about 3.9%, by weight, on a dry basis; a combination of catechin and epicatechin from about 20% to about 41%, by weight, on a dry basis; an epicatechin gallate (ecg) terminal unit percentage from about 0.9 to about 9.1; or an epicatechin gallate (ecg) extension unit percentage from about 1.4 to about 7.8.

In another aspect, the present disclosure provides a method of treating a subject, the method comprising administering to the subject a grape seed extract composition as described herein, wherein the grape seed extract composition is administered in an amount effective to improve cognition of the subject. In some embodiments, the grape seed extract composition is administered at about 100 mg/day to about 3000 mg/day, about 100 mg/day to about 2000 mg/day, about 100 mg/day to about 1000 mg/day, about 100 mg/day to about 500 mg/day, about 100 mg/day to about 300 mg/day, about 200 mg/day to about 3000 mg/day, about 400 mg/day to about 3000 mg/day, about 800 mg/day to about 3000 mg/day, about 1000 mg/day to about 3000 mg/day, about 1500 mg/day to about 3000 mg/day, or about 2000 mg/day to about 3000 mg/day. In particular embodiments, the grape seed extract composition is administered at about 300 mg/day to about 1000 mg/day. In certain embodiments, the grape seed extract composition is administered in a capsule, a tablet, a liquid formulation, a lozenge, or a powder. In other embodiments, administration occurs about every 12 hours, about every 24 hours, about every 36 hours, about every 48 hours, about once weekly, about twice weekly, about every two weeks, about every three weeks, or about once every month. In some embodiments, the subject is a human subject.

In yet another aspect, the present disclosure provides a grape seed extract composition produced by: obtaining a grape seed extract source material; filtering the grape seed extract source material to obtain a first retentate and a first permeate; drying the first retentate to obtain a first grape seed extract comprising a first mean degree of polymerization; filtering the first permeate to obtain a second retentate and a second permeate; drying the second retentate to obtain a second grape seed extract comprising a second mean degree of polymerization less than the first mean degree of polymerization; purifying the second permeate to obtain a purified liquid; drying the purified liquid to obtain a third grape seed extract comprising a third mean degree of polymerization less than the second mean degree of polymerization; and combining the first, second, or third grape seed extracts. In some embodiments, the grape seed extract composition comprises about 0-95 wt % of the first grape seed extract, about 0-95 wt % of the second grape seed extract, or about 0-95 wt % of the third grape seed extract, wherein a total percentage of the first, second, and third grape seed extracts equals 100%. In other embodiments, the grape seed extract composition comprises about 25-75 wt % of the first grape seed extract, about 15-45 wt % of the second grape seed extract, and about 2-20 wt % of the third grape seed extract, wherein a total percentage of the first, second, and third grape seed extracts equals 100%. In some embodiments, the grape seed extract composition comprises about 25-75 wt % of the first grape seed extract, about 15-45 wt % of the second grape seed extract, and about 2-20 wt % of the third grape seed extract, wherein a total percentage of the first, second, and third grape seed extracts equals 100%. In further embodiments, the composition comprises about 25-35 wt % of the first grape seed extract in combination with about 40-45 wt % of the second grape seed extract, and about 20-30 wt % of the third grape seed extract; about 35-45 wt % of the first grape seed extract, about 30-40 wt % of the second grape seed extract, and about 10-20 wt % of the third grape seed extract; about 45-55 wt % of the first grape seed extract, about 25-35 wt % of the second grape seed extract, and about 5-15 wt % of the third grape seed extract; about 55-65 wt % of the first grape seed extract, about 20-30 wt % of the second grape seed extract, and about 2-10 wt % of the third grape seed extract; or about 65-75 wt % of the first grape seed extract, about 15-25 wt % of the second grape seed extract, and about 2-8 wt % of the third grape seed extract. In particular embodiments, the composition comprises about 54 wt % of the first grape seed extract, about 34 wt % of the second grape seed extract, and about 12 wt % of the third grape seed extract. In some aspects, the composition improves the cognition of a subject when delivered in an effective amount to said subject; and in certain embodiments, the subject exhibits improvements on a standardized cognition test. In particular embodiments, the standardized cognition test is selected from the group consisting of Montreal Cognitive Assessment, Mini-Mental State Examination, Mini-Cog, and General Practitioner Assessment of Cognition.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a block diagram of a system for producing grape seed extracts, in accordance with certain embodiments.

FIG. 2A is a schematic diagram of an adsorption-elution column being operated in a fluidized condition, in accordance with certain embodiments.

FIG. 2B is a schematic diagram of an adsorption-elution column being operated in a packed condition, in accordance with certain embodiments.

FIG. 3 is a flowchart of a method of processing a grape seed extract, in accordance with certain embodiments.

FIGS. 4A-4C are chromatograms for three respective grape seed extracts, GSE A (FIG. 4A), GSE B (FIG. 4B), and GSE C (FIG. 4C), obtained using hydrophilic interaction liquid chromatography (HILIC) with a diode-array detector (DAD), at 280 nm.

FIG. 5 is a block diagram of a system for producing grape seed extracts, in accordance with certain embodiments, in accordance with certain examples.

FIG. 6 is a flowchart of a method of processing a grape seed extract, in accordance with certain embodiments.

FIG. 7 is a chromatogram for a grape seed extract obtained using hydrophilic interaction liquid chromatography (HILIC) with a diode-array detector (DAD), at 280 nm, in accordance with certain examples.

### DETAILED DESCRIPTION

The invention allows for the processing of a grape seed extract and further provides grape seed extracts produced from the methods and systems described, representing a significant advance over the prior art. In particular, the processes provided herein allow for the production of unique grape seed extracts enriched with high monomer (e.g. 20-40% catechin/epicatechin by weight), high oligomer, or high polymer compositions. The extracts can possess differing levels of astringency, taste and various biological activities (e.g., antioxidant, anti-inflammatory, blood pressure lowering effect) based on their characteristics described herein. For example, the grape seed extracts described herein may comprise higher or lower monomer levels compared with grape seed extracts known in the art.

The methods and systems provided herein may use only water and ethanol; and, in some embodiments, combine sequential membrane filtration, resin purification, and refractance window drying (RWD) to produce compositionally unique grape seed extracts. In contrast, other methods rely on solvents such as n-butyl acetate or ether for selective purification of procyanidins and subsequent drying by thin film evaporation (US20070078261A1) or rely on potentially hazardous techniques (U.S. Pat. No. 6,544,581B1) or toxic solvents (U.S. Pat. No. 7,566,401B2); and are therefore not readily scalable or economically viable. Unlike these other processes, the methods, systems, and starting materials described herein are practical in a production setting, improve operator safety (explosion hazards potentially eliminated during RWD), minimize environmental impacts (no highly toxic chemicals used, no hazardous waste generated), and have lower energy requirements.

The following is a detailed description provided to aid those skilled in the art in practicing the embodiments disclosed herein. Modifications and variations to the embodiments described herein can be made without departing from the spirit or scope of the present disclosure.

Apparatus, systems, and methods are provided for processing a grape seed extract, which may include one or more steps of, obtaining, drying, filtering, concentrating, purifying, or separating the grape seed extract as described herein. It is contemplated that the apparatus, systems, methods, and compositions disclosed herein encompass variations and adaptations developed using information from the embodiments described herein. Adaptation and/or modification of the apparatus, systems, methods, and compositions described herein may be performed by those of ordinary skill in the relevant art. It is also understood that the features of the various embodiments described herein are not mutually exclusive and can exist in various combinations and permutations.

Any embodiment discussed herein with respect to one aspect of the disclosure applies to other aspects as well, unless specifically noted.

As used herein, “degree of polymerization” or “DP” refers to a number of monomer subunits in a molecule, such as a macromolecule, an oligomer molecule, or a polymer molecule. For example, a molecule having DP >10 can be considered a polymer, a molecule having 2≤DP ≤10 can be considered an oligomer, and a molecule having DP=1 can be considered a monomer.

As used herein, “mean degree of polymerization” or “mDP” can refer to a mean or average degree of polymerization for the molecules in a composition. For example, a composition containing an equal mixture of two types of molecules, with one molecule having DP=2 and the other molecule having DP=4 can have mDP=3. Additionally, the mDP can be represented as a decimal number, including but not limited to 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.3, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, and 1.0. Standard techniques known in the art may be used to calculate mean degree of polymerization, terminal, and extension unit compositions. See Mattivi, F., et. al., Australian Journal of Grape and Wine Research 2009, 15, 25-35.

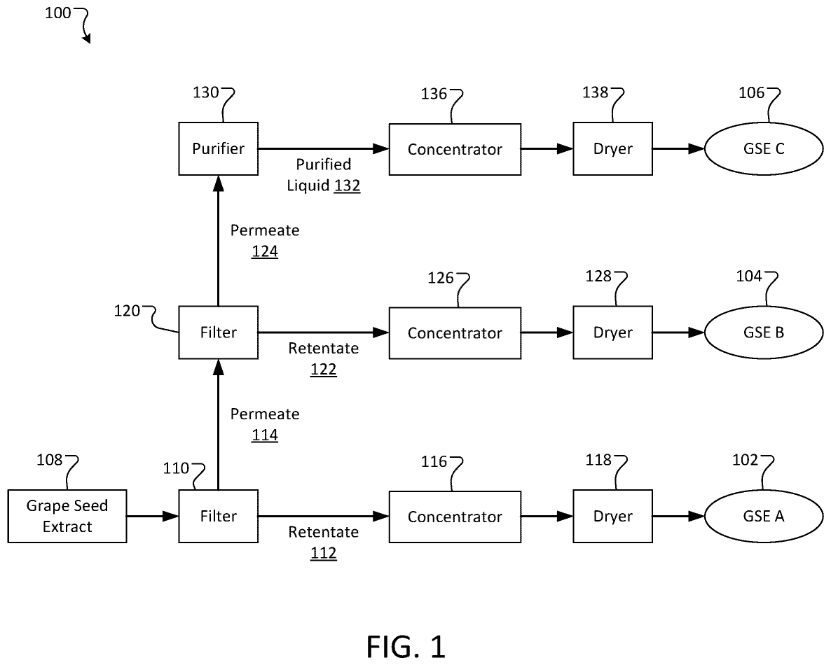

FIG. 1 depicts a block diagram of a system 100 for producing grape seed extract (GSE) compositions 102, 104, and 106 from a GSE source material 108, in accordance with the present disclosure. The GSE source material 108 can be or include an aqueous or alcoholic (e.g., ethanol) solution containing GSE. In the illustrated embodiments, the GSE source material 108 can be produced in a hot-water extraction process in which dry or fresh grape seeds are exposed to hot water (e.g., from about 140° F. to about 225° F., at pressures at or above atmospheric, for about 1 to 6 h) to extract polyphenols or other compounds from the seeds. The extracted materials can be filtered from spent seeds using metal screens. The aqueous extract may be cooled and/or treated with a pectinase enzyme (e.g., PECTINEX ULTRA SP-L; Novozyme) to break down extraneous materials such as cell wall constituents (e.g., pectin). The resulting extract may optionally be acidified with an acid (e.g., a mineral acid, an organic acid, or a food-grade acid, such as acetic acid, sulfuric acid, or phosphoric acid) to lower the pH from about 4 (e.g., 3.5 to 4.5) to about 2 (e.g., 1.5 to 2.5). Additionally or alternatively, tannase (e.g. from Kikkoman Biochemifa), an esterase, can be added to the extract to degrade one or more tannins (e.g., galloylated proanthocyanidins, gallotannins, and/or ellagitannins) and/or improve the solubility. The tannase may cleave gallic acid from the one or more tannins. Solids may be removed with a centrifuge (e.g., at 9000 rpm), a decanter, and/or by passing the liquid (or supernatant) through a microfilter (e.g., a dead-end filter (e.g., a WHATMAN 11 micron #1 filter, or a sock filter), a pressure filter (e.g., plate filter), a vacuum filter (e.g., rotary drum), a centrifugal filter, a cross-flow tubular membrane, a spiral wound membrane, or a ceramic membrane), to produce the GSE source material 108. Filtering aids (e.g., diatomaceous earth, perlite, cellulose fibers, paper pulp, etc.) may be employed, for example, in conjunction with dead-end filters to produce the GSE source material 108. Example processes for producing grape seed extract source materials are described in U.S. Pat. Nos. 6,544,581 and 8,075,929, the entire disclosure of each of which is incorporated by reference herein. In alternative embodiments, the GSE source material 108 can be produced from grape skins in addition to or instead of grape seeds, using the techniques described herein. Alternatively or additionally, the GSE source material 108 may be produced using a fermentation process or a precipitation process.

Exemplary characteristics of GSE source materials 108 are presented in Table 1, below. In general, a liquid form of the GSE source material can include a total amount of phenolics from about 6,000 ppm to about 350,000 ppm, a percent solids from about 0.6% to about 35%, a turbidity from about 3.3 NTU to about 32,400 NTU, a pH from about 2.0 to about 5.0, a total amount of catechin plus epicatechin from about 500 ppm to about 1500 ppm (or from about 5% to about 30% by weight, on a dry weight basis), and/or gallic acid from about 40 ppm to about 650 ppm (or from about 0.1% to about 8% by weight, on a dry weight basis).

TABLE 1
Example compositions for the GSE source material 108.
GSE
Source

| Material | | | % | Turbidity | | Catechin + | | Gallic | |
|---|---|---|---|---|---|---|---|---|---|
| 108 | Total Phenolics | | Solids | (NTU) | pH | Epicatechin | | Acid | |
| Example A | 6,000-9,000 | ppm | 2.8 | 32,400 | 3.8-4.8 | 500-1500 | ppm | 40-400 | ppm |
| | (liquid) | | | | | (liquid) | | (liquid) | |
| Example B | 3,000-9,000 | ppm | 0.6 | 3.3 | 2.0-3.0 | 500-1500 | ppm | 40-400 | ppm |
| | (liquid) | | | | | (liquid) | | | |
| Example C | 10,000-20,000 | ppm | 1.3 | 27 | 3.2 | 1309 | ppm | 650 | ppm |

(liquid)

| | | | | | | | |
|---|---|---|---|---|---|---|---|
| Example D | 250,000-350,000 | ppm | 25-35 | <30 | 2.5-5.0 | 5-30% | 0.1-8% |

| | | | |
|---|---|---|---|
| | (liquid) | (dry weight) | (dry weight) |
| Example E | 40-100% | 5-30% | 0.1-8% |
| | (dry weight) | (dry weight) | (dry weight) |

Still referring to FIG. 1, the GSE source material 108 can be provided to a filter 110, which can be used to produce a retentate 112 and a permeate 114. The filter 110 can be or include, for example, a membrane or an ultrafiltration filter, such as a spiral-wound membrane (e.g., 4.6 cm by 30.5 cm), a tubular membrane, and/or a ceramic membrane, where the liquid or filtrate can move tangentially with respect to the membrane (e.g., crossflow filtration). In some examples, the filter 110 can be incorporated into a high pressure, reverse osmosis system (e.g., an RO MINI, provided by TANGENT MEMBRANES, Inc.). The filter 110 can have a molecular weight cutoff from about 50,000 Daltons to about 300,000 Daltons, from about 80,000 Daltons to about 200,000 Daltons, or about 100,000 Daltons. In the illustrative embodiment, providing the GSE source material 108 to the filter 110 produces a retentate 112 and a permeate 114.

Other techniques can be used to produce the retentate 112 and the permeate 114 from the source material 108. For example, the retentate 112 and the permeate 114 can be produced using: dead-end filtration with a molecular weight cutoff, nanofiltration, liquid chromatography (e.g., reverse phase, normal phase, hydrophilic interaction, adsorption-elution, size-exclusion, etc.), counter-current chromatography, and/or supercritical fluid chromatography.

In general, the permeate 114 can include components having molecular weights below the molecular weight cutoff of the filter 110 (e.g., oligomers and monomers), and the retentate 112 can include components having molecular weights above the molecular weight cutoff of the filter 110 (e.g., polymers). For example, the filter 110 can prevent components having molecular weights above the cutoff from passing through the filter 110. In some embodiments, diafiltration can be performed (e.g., with water) to assist with the formation of the permeate 114 and the retentate 112, for example, to help carry lower molecular weight components through the filter 110 and into the permeate 114.

The filtration process (e.g., including ultrafiltration, nanofiltration, and/or diafiltration) can be performed at a temperature from about 80° F. (26.7° C.) to about 110° F. (43.3° C.), or about 95° F. (35° C.), and at pressure from about 25 psi (0.17 MPa) to about 75 psi (0.52 MPa), or about 50 psi (0.34 MPa). The temperature and/or pressure can be varied, if desired, to achieve more or less passage of materials through the filter 110 and thus can be used to adjust a final product composition. In some embodiments, the retentate 112 can be thermally or non-thermally pasteurized using, for example, flash or high-temperature short-time pasteurization, higher heat shorter time, retort, high pressure processing, UV light, ultrasound, pulsed electric fields, a plate and frame heat exchanger, or refractance window drying.

The retentate 112 produced using the filter 110 can be dried or de-watered using a concentrator 116 and a dryer 118. In general, the concentrator 116 can evaporate liquid (e.g., water) from the retentate 112 to increase a percent solids, which can enable a more efficient throughput and can assist with a drying process in the dryer 118. The concentrator 116 can evaporate the liquid at low pressure and/or can utilize a variety of techniques, including, for example, thin film vacuum distillation with heat (e.g., performed using a BÜCHI ROTAVAPOR, such as R-220 SE, R-210, or R-215), blow down, distillation, falling film, flooded, spinning cone, centrifugal, spontaneous, wiped film, other concentration techniques, or any combination thereof. The evaporated liquid can be recovered and/or recycled. The retentate 112 can have a percent solids content after the concentrator 116 from about 25% to about 50%. In certain embodiments, the retentate 112 can have a percent solids content after the concentrator 116 from about 33% to about 42%.

After the concentrator 116, the retentate 112 can be dried in the dryer 118, which can utilize a variety of drying techniques, including, for example, refractance window drying (RWD, alternatively referred to as conductance window drying or CWD), drum drying, conveyor drying, spray drying, or freeze drying. In certain embodiments, RWD is preferred because it is gentle and/or does not damage the retentate 112. This is in large part due to lower temperatures and shorter drying times used to remove water, thus minimizing product damage (e.g., oxidation, color loss, etc.). RWD can involve, for example, spreading the retentate 112 on a moving belt (e.g., a PET belt) disposed over a heated water bath. Water is removed from the retentate 112 as the belt travels through the dryer. Flakes of the dried mixture are removed from the belt at the dryer exit. The flakes can have a large range of particle sizes, which can be homogenized or reduced through milling, grinding, sieving, or other techniques. RWD technology is described in U.S. Pat. No. 11,221,179, issued on Jan. 11, 2022, the entire disclosure of which is incorporated by reference herein. Alternatively, the use of spray drying can be disadvantageous because, for example, a higher ratio of water to solids may be needed to ensure proper atomization, different particle sizes or high-bulk densities may not be achievable, and there can be high initial investment costs. Freeze drying 112 can be inefficient, energy intensive, and more expensive, compared to RWD. The dried material produced by the dryer 118 is the GSE composition 102, alternatively referred to herein as “GSE A.” The GSE composition 102 produced by the methods and systems described herein can comprise characteristics not found in grape seed extracts known in the art. For example, said composition (GSE A) can include a mean degree of polymerization from about 4.1 to about 5.3; and phenols in an amount from about 61 to about 70 g GAE/100 g, on a dry basis. In specific embodiments, said composition can include a mean degree of polymerization from about 4.3 to about 4.9; and phenols in an amount from about 63 to about 69 g GAE/100 g, on a dry basis. In still further embodiments, said composition can include a mean degree of polymerization of approximately 4.6; and phenols in an amount of approximately 67 g GAE/100 g, on a dry basis. In some embodiments, the GSE composition 102 can comprise gallic acid in an amount of approximately 0 on a dry basis; a combination of catechin and epicatechin from about 0 to about 0.5 on a dry basis; an epicatechin gallate (ecg) terminal unit percentage from about 0 to about 16; or an epicatechin gallate (ecg) extension unit percentage from about 5.6 to about 15.6.

Still referring to FIG. 1, the permeate 114 can be provided to a filter 120, which can be used to produce a retentate 122 and a permeate 124. The filter 120 can be or include, for example, a membrane or an ultrafiltration filter (or a nanofiltration filter), such as a spiral membrane (e.g., 4.6 cm by 30.5 cm), a spiral-wound cross-flow membrane, a tubular membrane, and/or a ceramic membrane (e.g., incorporated in a high pressure, reverse osmosis system). The filter 120 can have a molecular weight cutoff from about 300 Daltons to about 10,000 Daltons, from about 800 Daltons to about 5,000 Daltons, or about 1,000 Daltons. In some examples, instead of using a molecular weight cutoff of about 1,000 Da, membranes with larger cutoffs (e.g., 5,000 to 10,000 Daltons) or smaller cutoffs (e.g., 300 to 800 Daltons) can be used, for example, to achieve enrichment of monomers (e.g., achieved with a smaller cutoff), monomers and dimers (DP=2), or monomers, dimers, and trimers (e.g., achieved with a larger cutoff). In the illustrative embodiment, providing the permeate 114 to the filter 120 produces a retentate 122 and a permeate 124.

Other techniques can be used to produce the retentate 122 and the permeate 124 from the permeate 114. For example, the retentate 122 and the permeate 124 can be produced using: dead-end filtration with a molecular weight cutoff, nanofiltration, liquid chromatography (e.g., reverse phase, normal phase, hydrophilic interaction, adsorption-elution, size-exclusion, etc.), counter-current chromatography, and/or supercritical fluid chromatography.

In general, the permeate 124 can include components having molecular weights below the molecular weight cutoff of the filter 120 (e.g., monomers), and the retentate 122 can include components having molecular weights above the molecular weight cutoff of the filter 120 (e.g., oligomers). For example, the filter 120 can prevent components having molecular weights above the cutoff from passing through the filter 120. In some embodiments, diafiltration can be performed (e.g., with water) to assist with the formation of the permeate 124 and the retentate 122, for example, to help carry lower molecular weight components through the filter 120 and into the permeate 124. Diafiltration can increase an amount of monomer and/or gallic acid in the permeate 124 and/or can increase an amount of oligomer in the retentate 122.

The filtration process (e.g., including ultrafiltration, nanofiltration, and/or diafiltration) can be performed at a temperature from about 80° F. (26.7° C.) to about 110° F. (43.3° C.), or about 95° F. (35° C.), and at pressure from about 25 psi (0.17 MPa) to about 75 psi (0.52 MPa), or about 50 psi (0.34 MPa). The temperature and/or pressure can be varied, if desired, to achieve more or less passage of materials through the filter 120 and thus can be used to adjust a final product composition. In some embodiments, the retentate 122 can be thermally or non-thermally pasteurized using, for example, flash or high-temperature short-time pasteurization, higher heat shorter time, retort, high pressure processing, high pressure homogenization, UV light, ultrasound, pulsed electric fields, a plate and frame heat exchanger, or refractance window drying.

The retentate 122 produced using the filter 120 can be dried or de-watered using a concentrator 126 and a dryer 128. In general, the concentrator 126 can evaporate liquid (e.g., water) from the retentate 122 to increase a percent solids, which can enable a more efficient throughput and can assist with a drying process in the dryer 128. The concentrator 126 can evaporate the liquid at low pressure and/or can utilize a variety of techniques, including, for example, vacuum distillation with heat, blow down, distillation, falling film, flooded, spinning cone, centrifugal, spontaneous, wiped film, other concentration techniques, or any combination thereof. The evaporated liquid can be recovered and/or recycled. The retentate 122 can have a percent solids content after the concentrator 126 from about 25% to about 50%. After the concentrator 126, the retentate 122 can be dried in the dryer 128, which can utilize a variety of drying techniques, including, for example, RWD, drum drying, conveyor drying, spray drying, or freeze drying. In some embodiments, RWD is preferred because it is gentle and/or does not damage the retentate 122, as described herein. The dried material produced by the dryer 128 is the GSE composition 104, alternatively referred to herein as “GSE B.” The GSE composition 104 produced by the methods and systems described herein can comprise characteristics not found in grape seed extracts known in the art. For example, said composition (GSE B) can include a mean degree of polymerization from about 2.2 to about 2.7; and phenols in an amount from about 30 to about 60 g GAE/100 g, on a dry basis. In specific embodiments, said composition can include a mean degree of polymerization from about 2.3 to about 2.6; and phenols in an amount from about 40 to about 50 g GAE/100 g, on a dry basis. In still further embodiments, said composition can include a mean degree of polymerization of approximately 2.5; and phenols in an amount of approximately 40 g GAE/100 g, on a dry basis. In some embodiments, the GSE composition 104 can comprise gallic acid in an amount of about 0.3 to about 0.6 on a dry basis; a combination of catechin and epicatechin from about 0.3 to about 2.6 on a dry basis; an epicatechin gallate (ecg) terminal unit percentage from about 0 to about 14.4; or an epicatechin gallate (ecg) extension unit percentage from about 3.7 to about 11.5.

The permeate 124 can be dewatered (e.g., in a concentrator), if desired, and loaded into a purifier 130, which can be used to produce a purified liquid 132 having a higher concentration of condensed tannins, compared to the permeate 124. In some instances, the purifier 130 can be or include an adsorption-elution column (e.g., a glass, plastic, or metal column) containing a bed of resin particles for performing a fractionation process. The resin particles can be made of or include, for example, polyaromatic materials, divinylbenzene, polystyrene, methacrylate, acrylic ester, or other suitable materials. The resin particles may be functionalized with one or more moieties (e.g., cationic, anionic, bromine, etc.) or crosslinked to modify retention properties and final product composition. In some embodiments, the resin particles can include a macroporous resin that combines an aliphatic adsorbent resin structure with high surface area, such as XAD7HP resin (e.g., 300 mL BV) or FPX66 resin, available from DUPONT. As the permeate 124 passes through the column, condensed tannins can be adsorbed to the resin and more polar entities (e.g., anions, cations, sugars, organic acids, other impurities, etc.) can migrate past the resin and out of the column. The column can be washed with water to remove the polar entities. The purifier 130 can include any number of adsorption-elution columns (e.g., 1, 2, 4, 10, etc.) of any size.

Once the adsorption process is complete, the condensed tannins can be desorbed or eluted from the resin using a solvent, such as, for example, ethanol, methanol, propanol, iso-propanol, acetone, glycerol, glycol, ethyl acetate, and/or any solvent compatible with the resin or stationary phase. Such solvents may be used in any combination, with or without water, to effect selective desorption of certain chemistries. For example, ethanol (0-100%) can be used alone or in combination with water (0-100%) to selectively desorb the targeted chemistries described herein. The solvent used for desorption can be or include, for example, about 75% ethanol and about 25% water, by volume.

In some embodiments, the desorption process can utilize a step, linear, multilinear, or other gradient to vary a composition of the solvent and/or achieve a desired separation of chemistries. For example, in a step gradient fashion, 0-20% ethanol in water may be used to selectively desorb low molecular weight compounds (e.g., flavanol monomers, gallic acid, etc.). A solution of 20-50% ethanol in water can then be used to selectively desorb oligomers (e.g., tetramers, pentamers, etc.). Finally, a solution of 50-100% ethanol in water can be used to selectively desorb and elute oligomers and polymers. Other sequences of solvents or solvent concentrations can be used. In some instances, for example, a gradient elution may be employed where an ethanol concentration is increased and water concentration is simultaneously decreased (e.g., linearly), over time. Flowrates, solvent types, solvent concentrations, temperature, and/or time of exposure may be varied in any combination or sequence, to preferentially desorb and elute target or superfluous chemistries. A combination of step and gradient (e.g., linear gradient) may be employed.

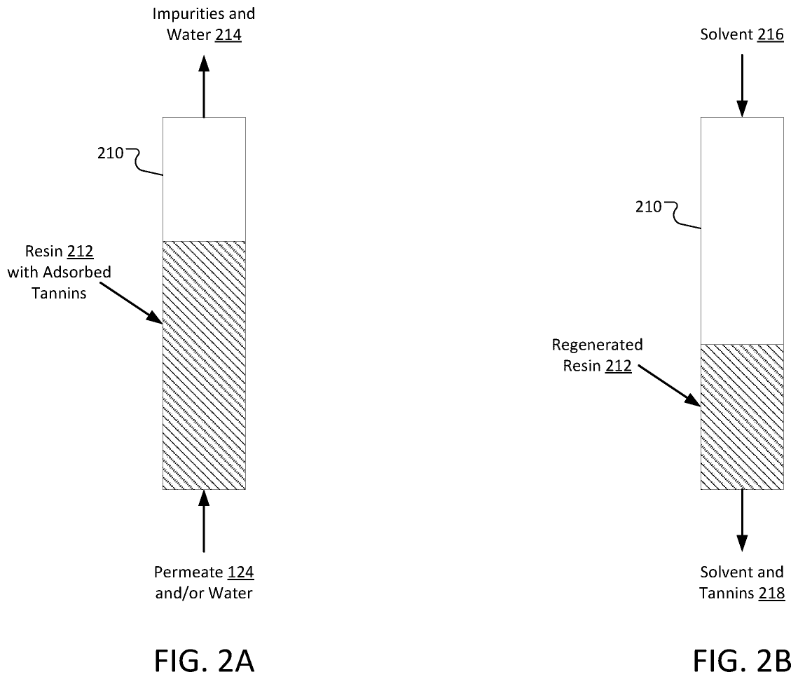

The adsorption-elution column can be operated in a packed or fluidized condition during the adsorption process and/or desorption process. In one illustrative embodiment, referring to FIG. 2A, an adsorption-elution column 210 can be operated in a fluidized condition during an adsorption process in which the permeate 124 is fed into the column 210 (or when the column is washed with water), tannins are adsorbed to a resin 212 in the column 210, and impurities and water 214 exit the column 210. The fluidized condition can be suitable when the permeate 124 includes particulates that could clog a column being operated in a packed condition (e.g., when the permeate 124 is cloudy or contains suspended particles). Additionally or alternatively, referring to FIG. 2B, the column 210 can be operated in a packed condition during a desorption process (or a resin regeneration process) in which a solvent 216 (e.g., ethanol) is passed through the resin 212 and the solvent and desorbed tannins 218 exit the column 210. In various embodiments, the adsorption and desorption processes can be performed using any number of columns 210, with each column 210 being operated in a packed condition and/or a fluidized condition.

Referring again to FIG. 1, in some embodiments, in addition to or instead of performing a fractioning process, the purifier 130 can utilize a variety of other techniques to purify the permeate 124. For example, membrane technologies such as nanofiltration, reverse-osmosis, or electrofiltration can be used in lieu of resin columns to purify target chemistries. Additionally or alternatively, chromatographic techniques can be utilized, such as flash, high-performance liquid, centrifugal partition, or counter-current. Such chromatographic techniques, however, can be expensive, have scaling limitations, utilize hazardous solvents, require specialized or experienced workers, or may not be environmentally friendly. In certain embodiments, the purifier 130 can utilize solvent-solvent partitioning, which can be performed in conjunction with counter-current techniques (e.g., centrifugal partition). Other possible techniques employed by the purifier 130 can include, for example, fermentation techniques (e.g., to convert trace fermentable sugars into carbon dioxide and ethanol), precipitation techniques (e.g., using polyamides, polyamines, aminopolysaccharides, aluminum chloride, or alkaloids as binding substances), and adsorptive bubble or foam fractionation. In some implementations, the purifier 130 (or a similar device) can be used to purify the retentate 112, the retentate 122, and/or the permeate 114 to enrich key chemistries (e.g., increase total phenols and/or a concentration of condensed tannins) in these materials.

In the illustrative embodiment shown in FIG. 1, providing the permeate 124 to the purifier 130 produces a purified liquid 132. The purified liquid 132 produced using the purifier 130 can be dried or de-watered using a concentrator 136 and a dryer 138. In general, the concentrator 136 can evaporate the solvent (e.g., ethanol) and/or other liquid (e.g., water) from the purified liquid 132 to increase a percent solids, which can enable a more efficient throughput and can assist with a drying process in the dryer 138. For example, the concentrator 136 can evaporate the solvent and water at low pressure and/or can utilize a variety of techniques, including, for example, vacuum distillation with heat, blow down, distillation, falling film, flooded, spinning cone, centrifugal, spontaneous, wiped film, other concentration techniques, or any combination thereof. The evaporated solvent (e.g., ethanol) can be recovered and/or recycled. The purified liquid 132 can have a percent solids content after the concentrator 136 from about 25% to about 50%.

After the concentrator 136, the purified liquid 132 can be dried in the dryer 138, which can utilize a variety of drying techniques, including, for example, RWD, spray drying, or freeze drying. In various embodiments, RWD is preferred because it is gentle and/or does not damage the purified liquid 132, as described herein. The dried material produced by the dryer 138 is the GSE composition 106, alternatively referred to herein as “GSE C.” The GSE composition 106 produced by the methods and systems described herein can comprise characteristics not found in grape seed extracts known in the art. For example, said composition (GSE C) can include a mean degree of polymerization from about 1.2 to about 1.4; and phenols in an amount from about 80 to about 95 g GAE/100 g, on a dry basis. In specific embodiments, said composition can include a mean degree of polymerization of approximately 1.3; and phenols in an amount of approximately 93 g GAE/100 g, on a dry basis. In some embodiments, the GSE composition 106 can comprise gallic acid in an amount of about 0.4 to about 3.9 on a dry basis; a combination of catechin and epicatechin from about 20 to about 41 on a dry basis; an epicatechin gallate (ecg) terminal unit percentage from about 0.9 to about 9.1; or an epicatechin gallate (ecg) extension unit percentage from about 1.4 to about 7.8.

In some embodiments, the system 100 may not utilize three dryers and/or three concentrators. For example, one dryer or two dryers can be used for the dryer 118, the dryer 128, and the dryer 138, such that two or all three of these dryers can be the same. Likewise, one concentrator or two concentrators can be used for the concentrator 116, the concentrator 126, and the concentrator 136, such that two or all three of these concentrators can be the same. In various embodiments, the system 100 or any component thereof can be operated in a continuous manner (e.g., a continuous process) or a discontinuous manner (e.g., a batch process).

In certain embodiments, the GSE compositions 102, 104, and 106 can be milled to achieve desired particle sizes or particle size distributions (e.g., to form a powder). For example, milling can be performed to achieve no more than 2% retained by a 40 mesh.

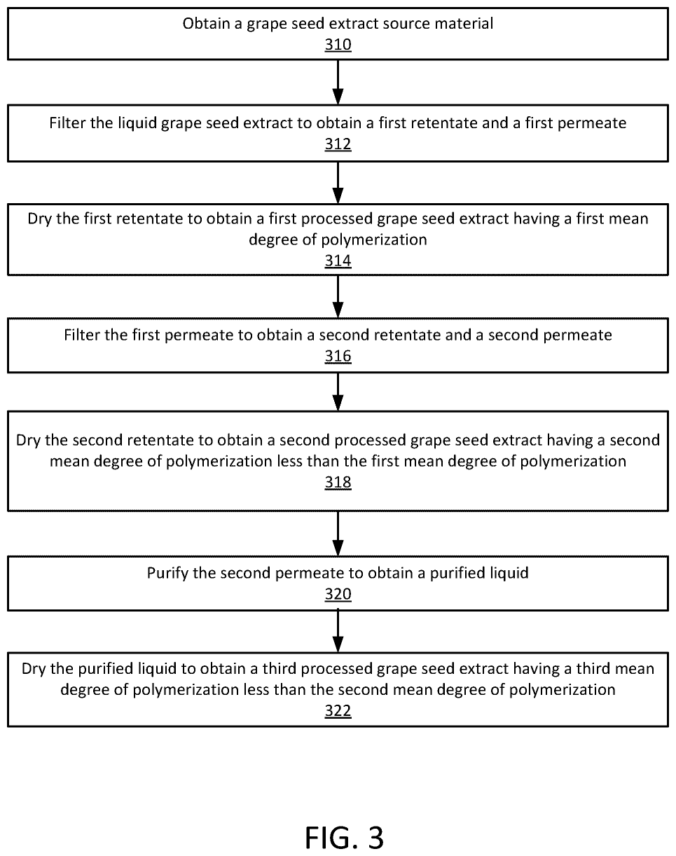

FIG. 3 is a flowchart of an exemplary method of processing a grape seed extract (e.g., using the system 100), in accordance with embodiments described herein. In method 300, a liquid grape seed extract is obtained (step 310) and filtered (step 312) to obtain a first retentate and a first permeate. The first retentate is dried (step 314) to obtain a first processed grape seed extract (e.g., GSE A) having a first mean degree of polymerization. The first permeate is filtered (step 316) to obtain a second retentate and a second permeate. The second retentate is dried (step 318) to obtain a second processed grape seed extract (e.g., GSE B) having a second mean degree of polymerization less than the first mean degree of polymerization. The second permeate is purified (step 320) to obtain a purified liquid. The purified liquid is dried (step 322) to obtain a third processed grape seed extract (e.g., GSE C) having a third mean degree of polymerization less than the second mean degree of polymerization.

Referring again to FIG. 1, in various embodiments, the system 100 and related methods described herein can utilize a sequence of filters 110 and 120 and the purifier 130 to separate the GSE source material 108 into the three GSE compositions 102, 104, and 106 (GSE A, GSE B, and GSE C, respectively). The chemistry and respective concentrations in these compositions 102, 104, and 106 can depend on how the GSE source material 108 was produced. In some embodiments, for example, the GSE source material 108 can be produced with or without tannase and with or without acid adjustment, such that there can be four different permutations for the GSE source material 108 (e.g., with tannase and with acid adjustment; with tannase and without acid adjustment; without tannase and with acid adjustment; and without tannase and without acid adjustment). These four permutations can result in four permutations for each of the three GSE compositions 102, 104, and 106.

For example, Tables 2 and 3 present experimental measurements of various parameters associated with the GSE compositions 102, 104, and 106 (GSE A, GSE B, and GSE C, respectively). Table 2 includes values for the compositions 102, 104, and 106 obtained when the GSE source material 108 was acidified (e.g., pH was about 2.2 to 2.4), with and without the use of tannase. Table 3 includes values for the compositions 102, 104, and 106 obtained when the GSE source material 108 was non-acidified (e.g., pH was about 4.1 to 4.3), with and without the use of tannase.

TABLE 2
Parameters for compositions 102, 104, and 106 (GSE A, B, and
C, respectively) obtained when the GSE source material 108 was
acidified to about 2.3, with and without the use of tannase.

| | Without Tannase | | | With Tannase | | |
|---|---|---|---|---|---|---|
| Parameter | GSE A | GSE B | GSE C | GSE A | GSE B | GSE C |
| Total Phenols (g GAE/100 g), Dry | 70% | 30% | 90% | 61% | 49% | 94% |
| Basis | | | | | | |
| wt % Gallic Acid, Dry Basis | ND | 0.4 | 3.9 | ND | 0.5 | 2.1 |
| wt % Total Catechin + | 0.4 | 0.3 | 36.5 | 0.5 | 0.6 | 29 |
| Epicatechin, Dry Basis | | | | | | |
| % epicatechin gallate (ecg) | 16 | 11.3 | 5.5 | 0 | 0 | 0.9 |
| terminal units | | | | | | |
| % epicatechin gallate (ecg) | 14.2 | 9.6 | 5.3 | 5.6 | 3.7 | 1.4 |
| extension units | | | | | | |
| Mean Degree of Polymerization | 4.3 | 2.2 | 1.2 | 4.1 | 2.6 | 1.4 |
| (mDP) | | | | | | |
| Water Activity (at about 22° C.) | 0.247 | 0.176 | 0.199 | 0.221 | 0.356 | 0.282 |
| % Moisture | 5.7 | 3.4 | 3.9 | 5.5 | 3.1 | 3.1 |
| pH of 1% Solution of | 3.1 | 2.3 | 3.1 | 3.1 | 2.4 | 3.3 |
| Composition in Water | | | | | | |
| % Yield of Total | 54 | 34 | 12 | 38 | 43 | 19 |

TABLE 3
Parameters for compositions 102, 104, and 106 (GSE A, B, and C,
respectively) obtained when the GSE source material 108 was non-
acidified (pH of about 4.2), with and without the use of tannase.

| | Without Tannase | | | With Tannase | | |
|---|---|---|---|---|---|---|
| Parameter | GSE A | GSE B | GSE C | GSE A | GSE B | GSE C |
| Total Phenols (g GAE/100 g), Dry | 67% | 47% | 93% | 70% | 34% | 95% |
| Basis | | | | | | |
| wt % Gallic Acid, Dry Basis | ND | 0.3 | 0.4 | ND | 0.6 | 1 |
| wt % Total Catechin + | 0.3 | 1.6 | 33.5 | 0.3 | 2.6 | 41 |
| Epicatechin, Dry Basis | | | | | | |
| % epicatechin gallate (ecg) | 15.9 | 14.4 | 9.1 | 7.2 | 4.2 | 2 |
| terminal units | | | | | | |
| % epicatechin gallate (ecg) | 15.6 | 11.5 | 7.8 | 10.3 | 4.8 | 2.1 |
| extension units | | | | | | |
| Mean Degree of Polymerization | 5.3 | 2.7 | 1.4 | 4.8 | 2.3 | 1.3 |
| (mDP) | | | | | | |
| Water Activity (at about 22° C.) | 0.389 | 0.296 | 0.274 | 0.245 | 0.257 | 0.246 |
| % Moisture | 8.69 | 2.72 | 3.28 | 5.43 | 3.5 | 2.99 |
| pH @ 1% | 3.99 | 3.91 | 4.19 | 3.78 | 4.06 | 4.04 |
| % Yield of Total | 57 | 29 | 14 | 58 | 30 | 12 |

As the experimental results in Tables 2 and 3 indicate, in general, the amount of total phenols (e.g., in grams of gallic acid equivalent (GAE) per 100 grams, dry basis) was highest in GSE C (from about 80% to about 99%), followed by GSE A (from about 50% to about 70%) and GSE B (from about 20% to about 60%). The results also indicate that, in general, the amount of gallic acid (e.g., in percent by weight, dry basis) was highest in GSE C (from about 1% to about 15%), followed by GSE B (from about 0% to about 5%) and GSE A (about 0% or not detected). The results also indicate that, in general, the amount of catechin plus epicatechin (e.g., in percent by weight, dry basis) was highest in GSE C (from about 10% to about 75%), followed by GSE B (from about 0.1% to about 5%) and GSE A (from about 0.1% to about 5%). The results in Tables 2 and 3 further indicate that tannase treated GSE compositions generally exhibit lower levels of epicatechin gallate (ecg) terminal units (from about 0% to about 2%) and epicatechin gallate (ecg) extension units (from about 0% to about 8%), compared to GSE compositions prepared without tannase. The results also indicate that tannase treated GSE compositions generally exhibit lower degree of galloylation (gallic acid esterification), as indicated by the amounts of epicatechin gallate terminal units and epicatechin gallate extension units, compared to GSE compositions prepared without tannase. The composition having the highest total epicatechin level was GSE C produced with tannase and a pH of about 4.2 (non-acidified). Otherwise, the compositions were not significantly influenced by pH adjustments.

For comparison, Table 4 presents experimental measurements of parameters associated with control compositions produced without using the filtration steps (e.g., at filters 110 and 120) and the purification step (e.g., at purifier 130). In this case, the four variations of GSE source material 108 used to generate the GSE compositions 102, 104, and 106 (i.e., with and without acidification and with and without tannase) were concentrated and dried (e.g., using the concentrator 116 and dryer 118, respectively).

TABLE 4
Parameters for control compositions produced from
GSE source material 108 with and without acidification
and with and without the use of tannase.

| | Without Tannase | | With Tannase | |
|---|---|---|---|---|
| | With | No | With | No |
| Parameter | Acid | Acid | Acid | Acid |
| Starting Liquid Extract pH | ~2 | ~4 | ~2 | ~4 |
| Total Phenols (g GAE/100 g), | 88% | 90% | 86% | 89% |
| Dry Basis | | | | |
| wt % Gallic Acid, Dry Basis | 1.2 | 1.1 | 4.8 | 2.8 |
| wt % Total Catechin + | 7.4 | 7 | 7.7 | 7.1 |
| Epicatechin, Dry Basis | | | | |
| % epicatechin gallate (ecg) | 10.9 | 11.1 | 0.7 | 4.1 |
| terminal units | | | | |
| % epicatechin gallate (ecg) | 11.2 | 11.2 | 3 | 8 |
| extension units | | | | |
| mDP | 2.7 | 2.6 | 2 | 2.7 |
| Water Activity | 0.21 | 0.26 | 0.22 | 0.24 |
| % Moisture | 5.3 | 5.39 | 4 | 4.61 |

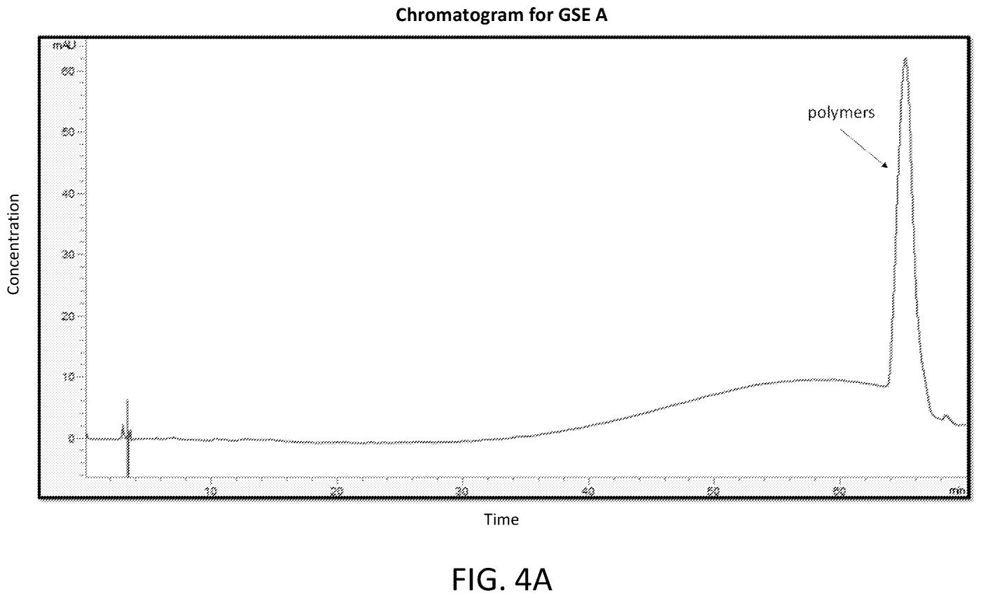

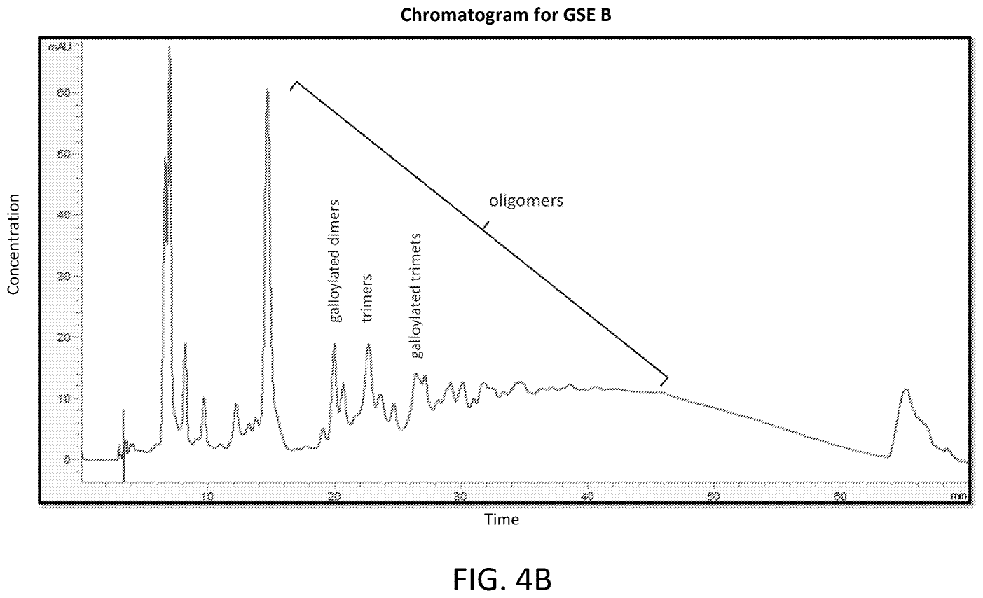

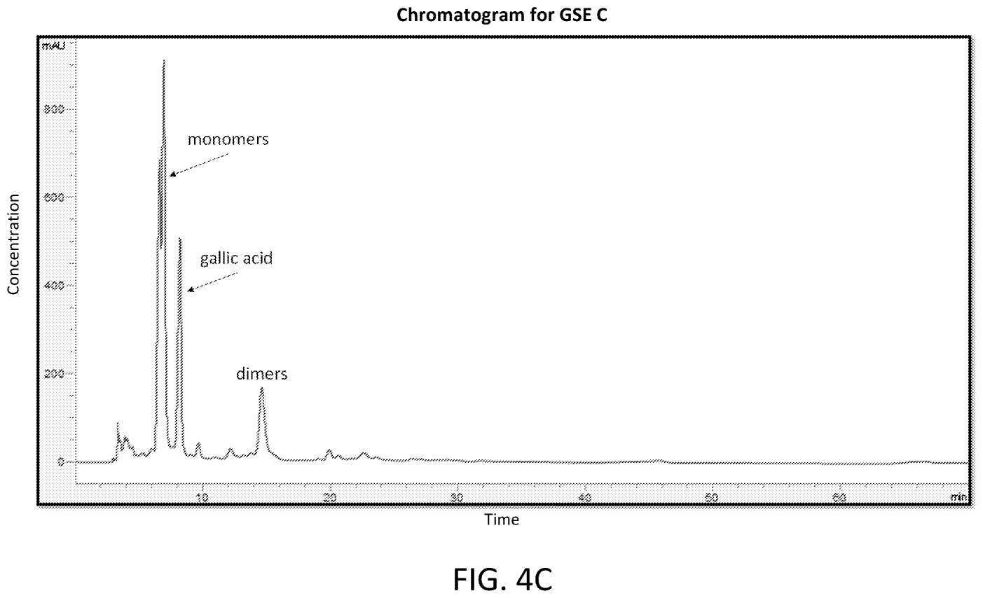

FIGS. 4A, 4B, and 4C depict chromatograms for exemplary GSE A, GSE B, and GSE C compositions, respectively, obtained using hydrophilic interaction liquid chromatography (HILIC) with a diol HPLC column (250×4.6 mm, 5 um particle size) and a diode-array detector (DAD) at 280 nm, in accordance with certain embodiments described herein. Hydrophilic interaction liquid chromatography (HILIC) is an alternative high-performance liquid chromatography (HPLC) mode for separating polar compounds. The chromatograms include peaks corresponding to respective components in each composition, with peaks for lower molecular weight components (e.g., monomers) appearing closer to a left-hand side of the x-axis and higher molecular weight components (e.g., polymers) appearing closer to a right-hand side of the x-axis. An area of each peak indicates a concentration or amount of the respective component that is present in the composition. Various components (e.g., “polymers,” “oligomers,” and “monomers”) that may be present in the compositions are labeled. As the figures indicate, GSE A in this example has a larger amount of polymers compared to GSE B and GSE C. GSE C in this example has larger amounts of monomers, gallic acid, and dimers, compared to GSE A and GSE B.

Tables 5 and 6 present experimental measurements of the relative amounts of monomers (e.g., monomeric flavanols, such as catechin and epicatechin), gallic acid, oligomers (e.g., oligomeric flavanols such as proanthocyanidins), and polymers (e.g., polymeric flavanols, such as proanthocyanidins) present in the GSE compositions 102, 104, and 106. The measurements were obtained using hydrophilic interaction liquid chromatography (HILIC) with a diol HPLC column (250×4.6 mm, 5 um particle size) and a diode-array detector (DAD) at 280 nm. Table 5 includes values for percentage of peak area in the HPLC measurements, for compositions 102, 104, and 106 (GSE A, B, and C, respectively) obtained when the GSE source material 108 was acidified (pH of about 2), with and without the use of tannase. Table 6 includes values for percentage of peak area in the HPLC measurements obtained when the GSE source material 108 was non-acidified (pH of about 4), with and without the use of tannase. In various embodiments, the values for percentage of peak area described herein may correspond to or be equal to weight percentages (wt %), on a dry basis.

TABLE 5
Example percentages of components for compositions 102,
104, and 106 (GSE A, B, and C, respectively) obtained
when the GSE source material 108 was acidified (pH
of about 2), with and without the use of tannase.

| | Without Tannase | | | With Tannase | | |
|---|---|---|---|---|---|---|
| Component | GSE A | GSE B | GSE C | GSE A | GSE B | GSE C |
| Monomers | 1.4 | 2.3 | 44.1 | 2.1 | 7.9 | 37.3 |
| Gallic Acid | 0 | 7.3 | 20.6 | 0 | 9.8 | 11.8 |
| Oligomers | 38.8 | 15.5 | 33.6 | 9.9 | 35.1 | 48.6 |
| Polymers | 59.8 | 74.9 | 1.7 | 88 | 47.2 | 2.3 |

TABLE 6
Example percentages of components for compositions 102,
104, and 106 (GSE A, B, and C, respectively) obtained
when the GSE source material 108 was non-acidified (pH
of about 4), with and without the use of tannase.

| | Without Tannase | | | With Tannase | | |
|---|---|---|---|---|---|---|
| Component | GSE A | GSE B | GSE C | GSE A | GSE B | GSE C |
| Monomers | 0.3 | 7.2 | 46.5 | 0.8 | 7.2 | 49.9 |
| Gallic Acid | 1 | 1.2 | 0.8 | 7.6 | 1.8 | 5.2 |
| Oligomers | 17.6 | 80.4 | 51.5 | 31.6 | 79.8 | 27.1 |
| Polymers | 81.1 | 11.2 | 1.2 | 60 | 11.2 | 17.8 |

As the results in Tables 5 and 6 indicate, in general and relative to one another, GSE A can have a highest percentage of polymers and lowest levels of monomers, GSE B can have a highest percentage of oligomers relative to other chemistries, and GSE C can have a highest percentage of monomers and gallic acid and lowest levels of polymers. The results also show that compositions produced using tannase can have higher levels of gallic acid, compared to compositions produced without using tannase.

Table 7 includes low, high, and typical values for various parameters associated with the compositions 102, 104, and 106 produced using the systems and methods described herein, in accordance with certain embodiments. The listed parameters can encompass possible ranges for the parameters that can be obtained depending on whether or not the GSE source material was prepared using tannase or acidification, as described herein. Each listed value can be a minimum, maximum, or average value (e.g., in a range of values). Various embodiments include any parameter value (e.g., integer or decimal value) within the cited ranges. For example, the total phenols value for GSE A can be greater than, less than, or equal to 61, 62, 63, . . . , or 70 g GAE/100 g. Express support and written description of these values for each parameter are hereby represented.

TABLE 7
Exemplary parameters for compositions 102, 104,
and 106 (GSE A, B, and C, respectively).

| | GSE A | | | GSE B | | | GSE C | | |
|---|---|---|---|---|---|---|---|---|---|
| Feature | Low | High | Typical | Low | High | Typical | Low | High | Typical |
| Total Phenols (g | 61 | 70 | 67 | 30 | 60 | 40 | 80 | 95 | 93 |
| GAE/100 g), Dry Basis | | | | | | | | | |
| wt % Gallic Acid, Dry | 0 | 0 | 0 | 0.3 | 0.6 | 0.45 | 0.4 | 3.9 | 1.9 |
| Basis | | | | | | | | | |
| wt % Total Catechin + | 0 | 0.5 | 0.4 | 0.3 | 2.6 | 1.3 | 20 | 41 | 35 |
| Epicatechin, Dry Basis | | | | | | | | | |
| % Epicatechin Gallate | 0 | 16 | 9.8 | 0 | 14.4 | 7.5 | 0.9 | 9.1 | 4.4 |
| (ecg) Terminal Units | | | | | | | | | |
| % Epicatechin Gallate | 5.6 | 15.6 | 11.4 | 3.7 | 11.5 | 7.4 | 1.4 | 7.8 | 4.2 |
| (ecg) Extension Units | | | | | | | | | |
| mDP | 4.1 | 5.3 | 4.6 | 2.2 | 2.7 | 2.5 | 1.2 | 1.4 | 1.3 |
| Monomers (% of | 0.3 | 2.1 | 1.2 | 2.3 | 7.9 | 6.2 | 37.3 | 49.9 | 44.5 |
| HPLC peak area) | | | | | | | | | |
| Oligomers (% of | 9.9 | 38.8 | 24.5 | 15.5 | 80.4 | 52.7 | 27.1 | 51.5 | 40.2 |
| HPLC peak area) | | | | | | | | | |
| Polymers (% of HPLC | 59.8 | 88.0 | 72.2 | 11.2 | 74.9 | 36.1 | 1.2 | 17.8 | 5.8 |
| peak area) | | | | | | | | | |
| Water Activity | 0.22 | 0.39 | 0.28 | 0.18 | 0.36 | 0.27 | 0.20 | 0.28 | 0.25 |
| % Moisture | 5.43 | 8.69 | 6.3 | 2.72 | 3.5 | 3.2 | 2.99 | 3.9 | 3.3 |
| pH @ 1% | 3.1 | 3.99 | 3.5 | 2.3 | 4.06 | 3.2 | 3.1 | 4.19 | 3.7 |
| % Yield of Total | 38 | 58 | 51.7 | 29 | 43 | 34.0 | 12 | 19 | 14.3 |

In various embodiments, the compositions 102, 104, and 106 (GSE A, B, and C, respectively) can be soluble (or substantially soluble) in water (e.g., distilled water) and/or ethanol (e.g., pure ethanol or 95% ABV). For example, any of the compositions 102, 104, and 106 can have a solubility in water or ethanol from about 0.001 g/mL to about 0.1 g/mL at room temperature.

The compositions 102, 104, and 106 (GSE A, B, and C, respectively) can have a wide variety of applications. For example, the compositions 102, 104, and 106 can be used as additives for food, beverages, confectionary, winemaking, cidermaking, brewing, spirits, food and beverage antioxidant supplements, food and beverage preservation, functional food and beverages, flavoring, confectionary, chocolate, dietary supplements, cosmetics, nutraceuticals, or medicinal applications. The compositions 102, 104, and 106 may be used as metal chelators and/or for removal of nitrogenous compounds, leather tanning, or functionalizing resins or membranes. In various implementations, the compositions 102, 104, and 106 can be used as additives in wine (e.g., Chardonnay or Pinot Noir), to improve taste or sensory perception. The monomers present in GSE C (e.g., catechin and/or epicatechin) can have a bitter flavor, compared to GSE A and GSE B, while GSE A and GSE B can be more astringent, compared to GSE C. Additionally or alternatively, the degree of galloylation can influence astringency and bitterness. For example, GSE B (or other composition) produced using tannase may have a lower degree of galloylation and less astringency, compared to GSE B produced without tannase, and may produce a less harsh but softer mouthfeel. Thus, the compositions described herein can serve as additives for wines (and other products), to effectively adjust bitterness or astringency and/or to produce wines that are well-rounded or have improved mouthfeel, structure, weight, viscosity, and/or body.

The terms, “effective”, “efficacious” or variations thereof are also terms interchangeably utilized in this application to describe the effects of grape seed compositions of the present invention. For example, an effective amount of the grape seed extract composition may refer to a quantity that, when administered to a subject according to methods described herein (e.g., as a capsule, tablet, liquid formulation, lozenge, gummy or powder at the recited dosing frequencies), yields a meaningful improvement in cognition in that subject relative to baseline or a control. In some embodiments, the improvement may be a statistically and/or clinically significant improvement as evidenced by enhancement of one or more validated cognitive endpoints (including, for example, memory, attention, or executive function), without undue adverse effects. Such amounts include, without limitation, daily doses of about 25 mg/day to about 3000 mg/day, including about 50 mg/day to about 2500 mg/day, and about 100 mg/day to about 1000 mg/day, administered in single or divided doses based on subject-specific factors to achieve the claimed cognitive improvement.

Additionally or alternatively, the compositions possess various biological activities (e.g., antioxidant, anti-inflammatory, and/or blood pressure lowering effects) and can provide a variety of health benefits. For example, the high oligomer content of GSE B can be beneficial for blood pressure, cardiovascular health, and/or antimicrobial or virucidal activity. The high polymer content in GSE A has demonstrated virucidal/antiviral characteristics. The high monomer content in GSE C can have a positive impact on human vascular health. One or more of the compositions can have a positive pre- and pro-biotic impact on gut health, which has implications for maladies associated with metabolic syndrome and cognitive health.

The compositions 102, 104, 106 (GSE A, B, and C, respectively), and combinations thereof can provide beneficial effects based on their unique characteristics (e.g. Total Phenols (g GAE/100 g), Dry Basis, wt % Gallic Acid, Dry Basis, wt % Total Catechin+Epicatechin, Dry Basis, % Epicatechin Gallate (ecg) Terminal Units, % Epicatechin Gallate (ecg) Extension Units, or mDP). Other grape seed extract compositions known in the art do not comprise such characteristics or provide the technical benefits described herein.

Administering an effective amount of a composition described herein yields improvements in cognition. The present disclosure therefore provides the use of grape seed extract compositions in methods for improving cognition when delivered in an effective amount to a subject. In some embodiments, administering an effective amount of a grape seed extract composition described herein to a subject may enhance cerebral blood flow, facilitate neurogenesis via upregulation of brain-derived neurotrophic factor (BDNF) and related signaling pathways, or provide antioxidant and anti-inflammatory protection to neural tissues. In certain aspects, the invention encompasses pharmaceutical, nutraceutical, or dietary supplement formulations comprising effective amounts of the grape seed extracts described herein, optionally in combination with other neuroprotective agents or excipients. Such formulations may be administered orally or by other suitable routes to improve cognition of a subject, ameliorate symptoms associated with neurodegeneration, or counteract the deleterious effects of oxidative stress and inflammation in the central nervous system.

Improvements in cognition may be measured by any method known in the art. For example, cognitive improvement is quantified using one or more standardized cognition tests selected from the group consisting of the Montreal Cognitive Assessment (MoCA), Mini-Mental State Examination (MMSE), Mini-Cog, and General Practitioner Assessment of Cognition (GPCOG). In some embodiments, a baseline assessment may be obtained within a predefined window, followed by repeat assessments at prespecified intervals following the published administration and scoring procedures for the selected tests. The primary efficacy endpoint is defined a priori as change from baseline on the selected test(s), evaluated as absolute score change, standardized change (e.g., z-scores), and/or responder status, the latter prespecified as meeting a minimal clinically important difference or categorical shift.

The present disclosure therefore provides compositions for improving cognition, promoting cognitive health, and/or mitigating neurodegenerative processes. In some embodiments, said compositions may comprise a first grape seed extract, a second grape seed extract, a third grape seed extract, or a combination thereof (e.g. GSE A, B, and/or C). In other embodiments, said compositions may comprise a first grape seed extract and a second grape seed extract, a first grape seed extract and a third grape seed extract, or a second grape seed extract and a third grape seed extract. For example, the composition may comprise at least one, at least two, or all three of a first grape seed extract, a second grape seed extract, and a third grape seed extract. In some embodiments, one of the extracts is omitted (0 wt %), such that the composition comprises two of the extracts in any relative proportions that sum to 100 wt %, including, by way of example: about 1-99 wt % of the first grape seed extract and about 99-1 wt % of the second grape seed extract (with the third at 0 wt %); about 1-99 wt % of the first grape seed extract and about 99-1 wt % of the third grape seed extract (with the second at 0 wt %); or about 1-99 wt % of the second grape seed extract and about 99-1 wt % of the third grape seed extract (with the first at 0 wt %), including proportions such as about 10/90, 25/75, 33/67, 40/60, 45/55, 50/50, 55/45, 60/40, 67/33, 75/25, or 90/10 wt %. In certain embodiments, a single extract may constitute substantially all of the composition (e.g., about 90-100 wt %, including about 100 wt %), with the other one or two extracts each at about 0-10 wt % (including about 0 wt %).

In particular embodiments, the composition for improving cognitive health and/or mitigating neurodegenerative processes comprises about 25-75 wt % of said first grape seed extract, about 15-45 wt % of said second grape seed extract, and about 2-20 wt % of said third grape seed extract, wherein the total percentage of said first grape seed extract, B, and C equals 100%. In non-limiting embodiments, suitable compositions may include, for example, approximately 25-35 wt % of said first grape seed extract in combination with approximately 40-45 wt % of said second grape seed extract and approximately 20-30 wt % of said third grape seed extract; approximately 35-45 wt % of said first grape seed extract, approximately 30-40 wt % of said second grape seed extract, and approximately 10-20 wt % of said third grape seed extract; approximately 45-55 wt % of said first grape seed extract, approximately 25-35 wt % of said second grape seed extract, and approximately 5-15 wt % of said third grape seed extract; approximately 55-65 wt % of said first grape seed extract, approximately 20-30 wt % of said second grape seed extract, and approximately 2-10 wt % of said third grape seed extract; and approximately 65-75 wt % of said first grape seed extract, approximately 15-25 wt % of said second grape seed extract, and approximately 2-8 wt % of said third grape seed extract. In preferred embodiments, the composition comprises about 54 wt % of said first grape seed extract, about 34 wt % of said second grape seed extract, and about 12 wt % of said third grape seed extract.

In some embodiments, the present disclosure provides methods wherein an effective amount of the grape seed extract composition comprises 25 mg/day to about 3000 mg/day, about 25 mg/day to about 2000 mg/day, about 25 mg/day to about 1000 mg/day, about 25 mg/day to about 500 mg/day, about 25 mg/day to about 300 mg/day, about 50 mg/day to about 3000 mg/day, about 250 mg/day to about 3000 mg/day, about 500 mg/day to about 3000 mg/day, about 1000 mg/day to about 3000 mg/day, about 1500 mg/day to about 3000 mg/day, about 2000 mg/day to about 3000 mg/day. The amounts of the grape seed extracts described herein, may also be expressed on a per-dose, per-day, per-week, or per-cycle basis. The foregoing ranges are inclusive of endpoints and encompass all integer and non-integer values and sub-ranges selected independently within the stated bounds. Doses may be adjusted based on route of administration, formulation, or subject weight. In certain embodiments, an effective amount of a grape seed extract composition as described herein may be represented as a daily effective amount, a twice daily effective amount, a bi-daily effective amount, or a weekly effective amount.

A grape seed extract or composition of the present disclosure is meant to encompass a composition suitable for administration to a subject, such as a mammal, particularly a human subject. In general, a composition is sterile, and preferably free of contaminants that are capable of eliciting an undesirable response within the subject (e.g., the compound(s) in the composition are pharmaceutical grade). Compositions may be designed for administration to subjects via oral administration.

An effective amount of a grape seed extract to improve cognition may be administered to a subject using a variety of acceptable dosage forms and routes of administration. For example, the grape seed extract may be formulated as an oral capsule, tablet, liquid solution or suspension, lozenge, or powder, or food (e.g., juice, granola, smoothie, gummy, or candy) each comprising an effective amount of the extract in combination with one or more acceptable excipients, carriers, or diluents. Capsules and tablets may be prepared using conventional techniques such as direct compression, wet granulation, or encapsulation, and may include additional agents to enhance stability, palatability, or controlled release. Liquid formulations may be provided as aqueous or non-aqueous solutions, emulsions, or suspensions, optionally containing flavoring agents, sweeteners, or preservatives to facilitate oral administration. Lozenges may be manufactured by molding or compression and are designed to dissolve slowly in the oral cavity, thereby enabling buccal or sublingual absorption. Powdered formulations may be provided in bulk or in unit-dose aliquots for reconstitution with a suitable liquid or for direct oral ingestion. Powdered formulas may be encapsulated to mask taste, incorporate into food, minimize degradation, and/or improve bioavailability. GSE may be agglomerated or refractance window dried to increase particle size and/or decrease surface area allowing for better stability, solubility and flowability. The selection of dosage form and route of administration is determined based on factors such as subject preference, age, swallowing ability, desired onset of action, and other similar considerations.

Precise amounts of the compositions also depend on the judgment of the practitioner and are peculiar to each individual. Factors affecting dose include physical and clinical state of the patient, the route of administration, the intended goal of treatment (alleviation of symptoms versus cure) and the potency and stability of the particular extract or other therapies a subject may be undergoing.

As used herein, “subject” or “patient” refers to animals, including humans, who are treated with the grape seed extracts or compositions in accordance with the methods described herein.

In some embodiments, the grape seed extract composition may be administered alone or in combination with other agents, such as therapeutic agents or additional medications to address cognitive decline, behavioral problems, and other health conditions. The grape seed extract composition may be provided as a single agent or as part of a pharmaceutical composition comprising one or more additional agents, excipients, or carriers. In some embodiments, the grape seed extract composition is administered in a dose and schedule sufficient to achieve an effective concentration in the target tissue or cell population.

In some embodiments, the grape seed extract composition may be administered every 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h, 47 h, or 48 hours. In particular embodiments, the grape seed extract composition is administered in a dose and schedule sufficient to achieve an effective concentration in the target tissue or cell population.

In certain embodiments, the present disclosure contemplates the use of grape seed extract compositions in the treatment of a variety of diseases and conditions, wherein modulation of cerebral blood flow, neurogenesis, and related signaling pathways, and providing antioxidant and anti-inflammatory protection is therapeutically beneficial. For example, in specific embodiments, the compositions described herein have utility in the prevention, mitigation, or treatment of cognitive decline, including but not limited to the treatment of neurodegenerative disorders such as Alzheimer's disease, age-associated dementia, and other conditions characterized by increased oxidative stress and neuronal loss. The ability of these compositions to maintain or restore glutathione levels, reduce reactive oxygen species, and inhibit neuroinflammatory processes suggests broader applications in ameliorating maladies associated with metabolic syndrome, which is frequently accompanied by increased oxidative stress, chronic inflammation, and impaired neuronal function. In specific embodiments, the grape seed extracts or compositions thereof protect neuronal cells from oxidative stress-induced cell death mediated by glutathione depletion. The grape seed extracts or compositions thereof may be employed to improve cognitive performance, enhance neuroprotection, or counteract the deleterious effects of metabolic dysregulation on the central nervous system.

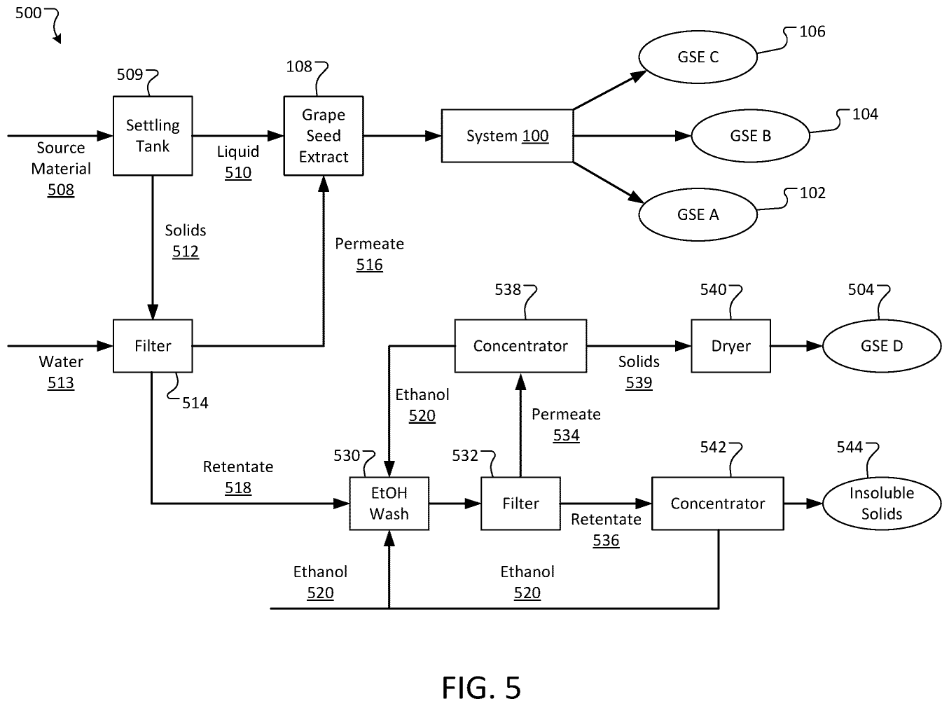

The present disclosure also provides a method and a system for processing a grape seed extract comprising: obtaining a mixture including a water-insoluble grape seed extract source material; adding ethanol to the mixture to form an ethanolic solution; filtering the ethanolic solution to obtain a permeate including ethanol and at least a portion of the water-insoluble grape seed extract source material dissolved in ethanol; and removing ethanol from the permeate to obtain a dried grape seed extract that is soluble in ethanol and insoluble in water. For example, FIG. 5 depicts a block diagram of a system 500 for producing a grape seed extract (GSE) composition 504 from a GSE source material 508, in accordance with examples described herein. The GSE source material 508 can be derived from grape seeds using a hot water extraction process (e.g., as described above for GSE source material 108), which may or may not utilize tannase and/or acidification, as described herein. The GSE source material 508 can be provided to a settling tank 509 and optionally chilled, to produce a liquid portion 510 (e.g., a water-soluble portion) and a solid portion 512 (e.g., a water-insoluble portion, including tank bottoms or heavy solids, with water-insoluble tannins). The liquid portion 510 can be the same as or can be used to produce the GSE source material 108, which can be used in the system 100 to produce GSE compositions 102, 104, and 106 (GSE A, B, and C, respectively), as described above.

In some embodiments, the solid portion 512 can be mixed with water 513, resuspended (e.g., with mixing), and separated using a filter 514 or other separation device to produce a permeate 516 and a retentate 518. The permeate 516 can be combined with the liquid portion 510 and/or used to produce the GSE source material 108. The filter 514 can be or include, for example, a dead-end filter (e.g., sock filter; 1-100 μm) membrane or an ultrafiltration filter, such as a spiral-wound membrane (e.g., 4.6 cm by 30.5 cm), a tubular membrane, and/or a ceramic membrane, where the liquid or filtrate can move tangentially with respect to the membrane (e.g., crossflow filtration). In some embodiments, the filter 514 can be incorporated into a high pressure, reverse osmosis system (e.g., an RO MINI, provided by TANGENT MEMBRANES, Inc.). The filter 514 can have a molecular weight cutoff from about 500,000 Daltons to about 700,000 Daltons, or about 600,000 Daltons. In certain embodiments, diafiltration can be performed (e.g., with water) to assist with the formation of the permeate 516 and the retentate 518, for example, to help carry lower molecular weight components through the filter 514 and into the permeate 516, and/or to concentrate or modify chemistries characteristic of the permeate 516 and retentate 518.

The retentate 518 can include water-insoluble solids and can be provided to an ethanol wash device 530 where the retentate 518 can be mixed with ethanol 520 or other suitable solvent. The wash device 530 can dissolve one or more components of the retentate 518 in the ethanol 520, which can be or include a mixture of ethanol and water and/or can have an ethanol concentration greater than about 90% ABV (e.g., about 95% ABV or 190 proof). To facilitate dissolution, the retentate 518 can be mixed with the ethanol 520 using a mixing device, such as a mixing blade or a homogenizer (e.g., an immersion homogenizer operated at 2500 rpm).

The resulting mixture of the retentate 518 and ethanol 520 is provided to a filter 532, which is used to produce an ethanol-soluble permeate 534 and an ethanol-insoluble retentate 536. The filter 532 can be or include a coarse filter (e.g., WHATMAN #1 filter paper), a filter sock, an ultrafiltration filter, and/or a centrifuge. In some embodiments, the filter 532 can have a pore size of about 0.4 microns and/or a molecular weight cutoff from about 150,000 Daltons to about 250,000 Daltons, or about 200,000 Daltons. The filter 532 can be resistant to degradation by ethanol (e.g., ethanol compatible). In certain embodiments, diafiltration can be performed (e.g., with ethanol or water) to assist with the formation of the permeate 534 and the retentate 536, for example, to help carry ethanol 520 or lower molecular weight components through the filter 532 and into the permeate 534, and/or to concentrate or modify chemistries characteristic of the permeate 534 and retentate 536.

The permeate 534 can be provided to a concentrator 538 where ethanol 520 can be removed from ethanol-soluble solids 539 (e.g., ethanol-soluble tannins) in the permeate 534. The concentrator 538 can utilize a variety of separation techniques. For example, the concentrator 538 can evaporate the ethanol 520 at low pressure and/or can utilize a variety of techniques, including, for example, vacuum distillation with heat, blow down, distillation, falling film, flooded, spinning cone, centrifugal, spontaneous, wiped film, other concentration techniques, or any combination thereof. Alternatively or additionally, the concentrator 538 can precipitate the ethanol-soluble solids 539 from the ethanolic solution by adding water (with or without chilling) followed by filtration (e.g., dead-end filtration) or centrifugation, as described herein. The ethanol 520 may be recovered, for example, by condensing ethanol vapor back to a liquid form (e.g., in a distillation process). The recovered ethanol 520 may be recycled and used in the ethanol wash device 530. The solids 539 after the concentrator 538 can have a percent solids content from about 25% to about 50%.

After the concentrator 538, the solids 539 can be dried in a dryer 540, which can utilize a variety of drying techniques, including, for example, RWD, spray drying, or freeze drying. In various embodiments, RWD is preferred because it is gentle and/or does not damage the solids 539, as described herein. The dried material produced by the dryer 540 is the GSE composition 504, alternatively referred to herein as “GSE D.” GSE D may be or include highly polymeric condensed tannins and/or may be largely devoid of monomers, oligomeric, and other low molecular weight compounds (e.g., gallic acid). GSE D may be soluble (or substantially soluble) in ethanol but insoluble (or substantially insoluble) in water. For example, GSE D may have a solubility in ethanol (e.g., pure ethanol or about 95% ABV) from about 0.001 g/mL to about 0.1 g/mL, at room temperature. By comparison, the solubility of GSE D in water (e.g., distilled water) may be less than about 0.0001 g/mL, less than about 0.00001 g/mL, or zero, at room temperature.

The retentate 536 produced by the filter 532 can be provided to a concentrator 542, which can evaporate ethanol 520 and/or other liquids (e.g., water) from the retentate 536. For example, the concentrator 542 can evaporate the ethanol at low pressure and/or can utilize a variety of techniques, including, for example, vacuum distillation with heat, blow down, distillation, falling film, flooded, spinning cone, centrifugal, spontaneous, wiped film, other concentration techniques, or any combination thereof. Additionally or alternatively, the concentrator 542 can utilize or include a dryer, which can use a variety of drying techniques, including, for example, RWD, spray drying, or freeze drying. The ethanol 520 can be recovered (e.g., by distillation or condensation) and recycled for use in the ethanol wash device 530. The concentrator 542 can output a solid material 544 that is insoluble (or substantially insoluble) in water and ethanol. The solid material 544 may be soluble (or substantially soluble) in dimethyl sulfoxide (DMSO), sulfuric acid (e.g., 10 mg/mL), sodium hydroxide (e.g., 10 mg/mL), or other solvents.

In various embodiments, the system 500 or any component thereof can be operated in a continuous manner (e.g., a continuous process) or a discontinuous manner (e.g., a batch process). Additionally or alternatively, equipment used in the system 500 can be explosion-proof and/or leak-proof to ensure safe handling of ethanol or other volatile or flammable materials. In certain implementations, a purifier (e.g., the purifier 130 or similar device) can be used to further process the permeate 516, the retentate 518, and/or the permeate 534, for example, to enrich key chemistries (e.g., increase total phenols and/or a concentration of condensed tannins) in these materials.

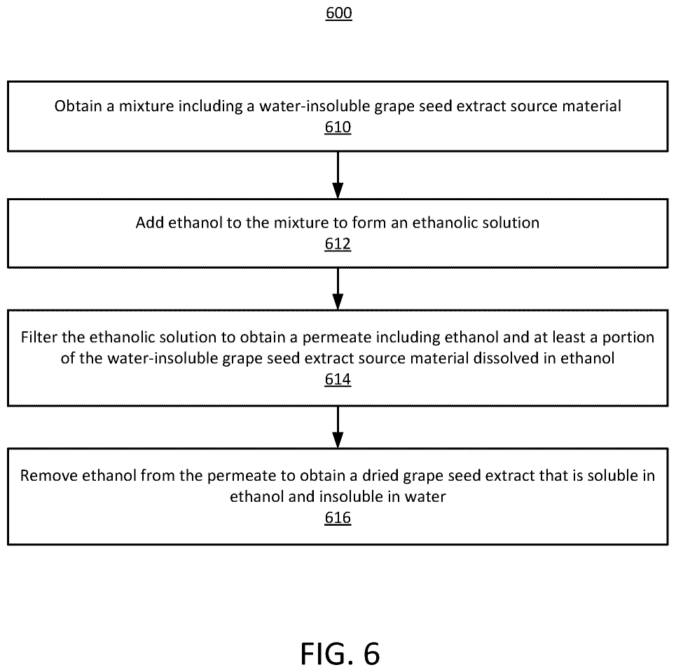

FIG. 6 is a flowchart of an example method 600 of processing a grape seed extract (e.g., using the system 500), in accordance with embodiments described herein. A mixture including a water-insoluble grape seed extract source material is obtained (step 610). Ethanol is added (step 612) to the mixture to form an ethanolic solution. The ethanolic solution is filtered (step 614) to obtain a permeate including ethanol and at least a portion of the water-insoluble grape seed extract source material dissolved in ethanol. Ethanol is removed (step 616) from the permeate to obtain a dried grape seed extract that is soluble in ethanol and insoluble in water (e.g., GSE D).

Table 8 presents experimental measurements of the relative amounts of monomers (e.g., monomeric flavanols, such as catechin and epicatechin), oligomers (e.g., oligomeric flavanols such as proanthocyanidins), and polymers (e.g., polymeric flavanols, such as proanthocyanidins) present in the GSE composition 504 (GSE D). In this particular embodiment, the GSE composition 504 was separated using hydrophilic interaction liquid chromatography (HILIC) with a diol HPLC column (250×4.6 mm, 5 um particle size) and a diode-array detector (DAD) at 280 nm. The values in the table indicate that GSE D comprises mostly polymers.

TABLE 8
Example percentages of components for composition 504 (GSE D).

| Component | GSE D |
|---|---|
| Monomers (% of HPLC peak area) | 3.2 |
| Oligomers (% of HPLC peak area) | 6.8 |
| Polymers (% of HPLC peak area) | 90 |

Table 9 presents experimental measurements of the amounts of total phenols (determined by Folin-Ciocalteu), (epi) catechin (determined by HPLC), and gallic acid (determined by HPLC), for the GSE composition 504 (GSE D).

TABLE 9
Example percentages of components for composition 504 (GSE D).

| Component | GSE D |
|---|---|
| Total Phenols (g GAE/100 g), Dry Basis | 67.6 |
| wt % Epicatechin (Dry Basis) | 0.6 |
| wt % Gallic Acid (Dry Basis) | 0.3 |

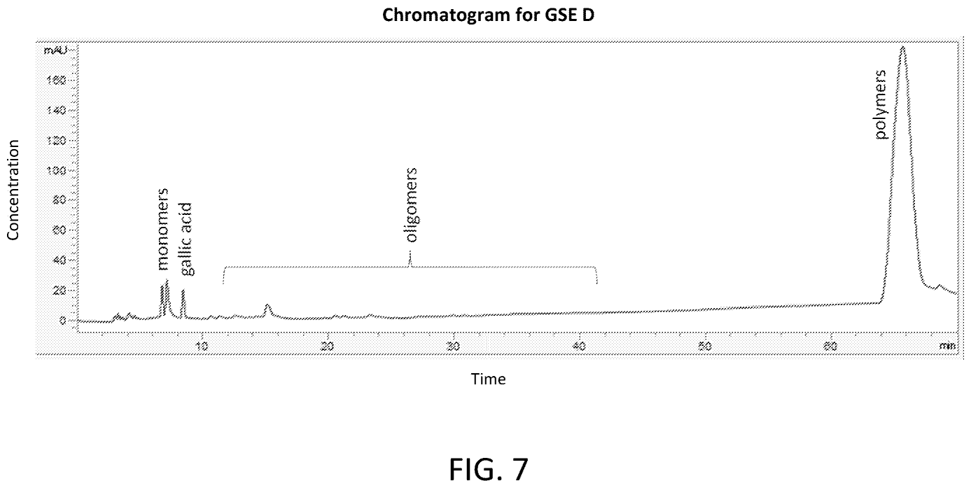

FIG. 7 depicts a chromatogram for an example of GSE D, obtained using hydrophilic interaction liquid chromatography (HILIC) with a diol HPLC column (250×4.6 mm, 5 um particle size) and a diode-array detector (DAD) at 280 nm, in accordance with certain embodiments described herein. The chromatogram includes peaks corresponding to respective components in each composition, with peaks for lower molecular weight components (e.g., monomers) appearing closer to a left-hand side of the x-axis and higher molecular weight components (e.g., polymers) appearing closer to a right-hand side of the x-axis. An area of each peak indicates a concentration or amount of the respective component that is present in the composition taking into consideration the molar absorptivity coefficient of the component of interest. As the figure indicates, GSE D in this example has a high polymer content.

Table 10 includes low, high, and typical values for various parameters associated with the composition 504 (GSE D) produced using the systems and methods described herein, in accordance with certain embodiments. The listed parameters can encompass possible ranges for the parameters that can be obtained depending on whether or not the GSE source material was prepared using tannase or acidification, as described herein. Each listed value can be a minimum, maximum, or average value (e.g., in a range of values). Various embodiments include any parameter value (e.g., integer or decimal value) within the cited ranges. For example, the total phenols value for GSE D can be greater than, less than, or equal to 55, 56, . . . , 74, or 75 g GAE/100 g. Express support and written description of these values for each parameter are hereby represented.

TABLE 10
Exemplary parameters for composition 504 (GSE D).

| | GSE D | | |
|---|---|---|---|
| Feature | Low | High | Typical |
| Total Phenols (g GAE/100 g), Dry Basis | 55 | 75 | 68 |
| wt % Gallic Acid, Dry Basis | 0 | 2 | 0.3 |
| wt % Total Catechin + Epicatechin, Dry | 0 | 2 | 0.6 |
| Basis | | | |
| % Epicatechin Gallate (ecg) Terminal | 0 | 6 | 3.3 |
| Units | | | |
| % Epicatechin Gallate (ecg) Extension | 0 | 12 | 9.1 |
| Units | | | |
| mDP | 3 | 7 | 4.4 |
| Monomers (% of HPLC peak area) | 0 | 5 | 3 |
| Oligomers (% of HPLC peak area) | 0 | 11 | 7 |
| Polymers (% of HPLC peak area) | 75 | 99 | 90 |
| Solubility in Distilled Water (g/mL at | 0 | 0.00001 | 0 |
| 21.6° C.) | | | |
| Solubility in Ethanol (g/mL at 21.6° C.) | 0.001 | 0.1 | 0.014 |
| Water Activity at 21.4° C. | 0.1 | 0.5 | 0.41 |
| % Moisture | 1 | 11 | 10.7 |

The GSE composition 504 (GSE D) produced using the system 500 can have a wide variety of applications. For example, the composition 504 can be used as an additive for food, beverages, confectionary, winemaking, cidermaking, brewing, spirits, food and beverage antioxidant supplements, food and beverage preservation, functional food and beverages, flavoring, confectionary, chocolate, metal chelators, leather tanning, dietary supplements, cosmetics, nutraceuticals, or medicinal applications. In some embodiments, the composition 504 (GSE D) can be used to produce wines with improved mouthfeel, body, structure, and/or astringency.

While much of the subject matter described herein relates to grape seed extracts, it is understood that the systems, methods, and compositions described herein can be used with or derived from other materials. For example, the systems and methods can be extended beyond grape seed materials to include fractionation of any natural or man-made source, extract, or fraction that contains mixtures of any of the following: proanthocyanidins or condensed tannins (e.g., procyanidins, propelargonidins and/or prodelphinidins), hydrolyzable tannins (e.g., gallotannins and/or ellagitannins), carbohydrates, oligosaccharides, polysaccharides (e.g., pectin), aminopolysaccharides (e.g., chitosans), oligonucleotides, polyamides (e.g., peptides or proteins), polyacrylamides, polyamines, polyacrylic acid copolymers, polysorbates, polyketides, poloxamers, polyethylene glycols (alternatively referred to as polyethylene oxide or polyoxyethylene alcohols), polyvinyl alcohols, macromolecular complexes (e.g., tannin-protein, tannin-polysaccharide, protein-polysaccharide, etc.), polymeric pigments (e.g., anthocyanin-ethyl-flavanols, pyranoanthocyanin-flavanol, anthocyanin-flavanol, polymerized anthocyanins, portisins, etc.), melanoidins, phlobaphenes, phlobatannins, (bio) synthetic derivatives, chemically modified, and any combinations thereof.

In various embodiments, the monomers present in the compositions (e.g., GSE A, B, C, and D) and materials (e.g., grape seeds, grape skins, and GSE source material 108) described herein can be or include (+) catechin, (−) epicatechin, (−) epicatechin-3-O-gallate, and/or (−) epigallocatechin gallate. The oligomers and polymers present in the compositions and materials described herein can include or be composed of any of these monomers, their isomers, alone or in any combination. For example, in heated aqueous solutions, (+)-catechin and (−)-epicatechin can epimerize to (+)-epicatechin and (−)-catechin, respectively.

### Definitions

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Each numerical value presented herein, for example, in a table, a chart, or a graph, is contemplated to represent a minimum value or a maximum value in a range for a corresponding parameter. Accordingly, when added to the claims, the numerical value provides express support for claiming the range, which may lie above or below the numerical value, in accordance with the teachings herein. Absent inclusion in the claims, each numerical value presented herein is not to be considered limiting in any regard.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention.

Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

### EXAMPLES

### Example 1—Production of GSE C with Acid Adjustment and without Tannase

In this example, GSE C was produced from a GSE source material (e.g., GSE 108) obtained with acidification (e.g., pH of about 2) and without tannase treatment. The GSE source material was filtered with an ultrafiltration filter (e.g., filter 120) having a molecular weight cutoff of 1,000 Daltons to produce a permeate (e.g., permeate 124). The permeate (about 44 L) was rotavaped using a BÜCHI R-220 SE at 65° C. and a vacuum pressure (about 40 mbar) to remove excess water down to a small volume (about 1.5 to 2 L). The concentrated permeate was loaded on a glass column (120×400 mm) packed with XAD7HP resin (300 mL BV) via a peristaltic pump (WATSON-MARLOW model 5205). Pump and elution flowrates were both about 24 mL/min. Following the loading of permeate, the column was washed with water (about 600 mL) to remove impurities (e.g., sugars, organic acids, salts, insoluble particulates, etc.). Tannins were desorbed from the resin and eluted from the column with ethanol (about 70-99%) at a flowrate of about 5 mL/min. Desorbed condensed tannins and ethanol were rotary evaporated to remove ethanol and concentrate using thin film evaporation under reduced pressure with BÜCHI rotavapors (e.g., R-210, R-215). Exemplary GSE compositions produced according to this method are described in Tables 2, 4, 5, and 7.

### Example 2—Production of GSE C without Acid Adjustment and without Tannase

In this example, GSE C was produced from a GSE source material (e.g., GSE 108) obtained without acidification (e.g., pH of about 4) and without tannase treatment. The GSE source material was filtered with an ultrafiltration filter (e.g., filter 120) having a molecular weight cutoff of 1,000 Daltons to produce a permeate (e.g., permeate 124). The permeate (about 41 L) was loaded on a glass column (120×400 mm) packed with XAD7HP resin (300 mL BV) via a peristaltic pump (WATSON-MARLOW model 5205). Pump and elution flowrates were both about 24 mL/min. Following permeate loading, the column was washed with water (about 600 mL) to remove impurities (e.g., sugars, organic acids, salts, insoluble particulates, etc.). Tannins were desorbed from the resin and eluted from the column with ethanol (about 70-99%) at a flowrate of about 5 mL/min. Desorbed condensed tannins and ethanol were rotary evaporated to remove ethanol and concentrate using thin film evaporation under reduced pressure with BÜCHI rotavapors (models R-210, R-215). Exemplary GSE compositions produced according to this method are described in Tables 3, 4, 6, and 7.

### Example 3—Production of GSE C with Acid Adjustment and with Tannase

In this example, GSE C was produced from a GSE source material (e.g., GSE 108) obtained with acidification (e.g., pH of about 2) and with tannase treatment. The GSE source material was filtered with an ultrafiltration filter (e.g., filter 120) having a molecular weight cutoff of 1,000 Daltons to produce a permeate (e.g., permeate 124). The permeate (about 41 L) was loaded on a glass column (120×400 mm) packed with XAD7HP resin (300 mL BV) via a peristaltic pump (WATSON-MARLOW model 5205). Pump and elution flowrates were both about 32 mL/min. Following the loading of permeate, the column was washed with water (about 600 mL) to remove impurities (e.g., sugars, organic acids, salts, insoluble particulates, etc.). Tannins were desorbed from the resin and eluted from the column with ethanol (about 70-99%) at a flowrate of about 5 mL/min. Desorbed condensed tannins and ethanol were rotary evaporated to remove ethanol and concentrate using thin film evaporation under reduced pressure with BÜCHI rotavapors (models R-210, R-215). Exemplary GSE compositions produced according to this method are described in Tables 2, 4, 5, and 7.

### Example 4—Production of GSE C without Acid Adjustment and with Tannase

In this example, GSE C was produced from a GSE source material (e.g., GSE 108) obtained without acidification (e.g., pH of about 4) and with tannase treatment. The GSE source material was filtered with an ultrafiltration filter (e.g., filter 120) having a molecular weight cutoff of 1,000 Daltons to produce a permeate (e.g., permeate 124). The permeate (about 42 L) was loaded on a glass column (120×400 mm) packed with XAD7HP resin (300 mL BV) via a peristaltic pump. Pump and elution flowrates were both about 32 mL/min. Following the loading of permeate, the column was washed with water (about 600 mL) to remove impurities (e.g., sugars, organic acids, salts, insoluble particulates, etc.). Tannins were desorbed from the resin and eluted from the column with ethanol (about 70-99%) at a flowrate of about 5 mL/min. Desorbed condensed tannins and ethanol were rotary evaporated to remove ethanol and concentrate using thin film evaporation under reduced pressure with BUCHI rotavapors (models R-210, R-215). Exemplary GSE compositions produced according to this method are described in Tables 3, 4, 6, and 7.

### Example 5—Production of GSE D

GSE D was produced from a GSE retentate obtained using an OMNIA microfilter having a pore size of about 0.2 um and a molecular weight cutoff of about 100K Daltons. About 3.8 L of the retentate was vacuum filtered through a BÜCHNER funnel and WHATMAN #1 filter paper. The clarified filtrate (about 3.25 L) was loaded into the top of a glass column charged with XAD17HP resin (500 mL BV). The filtrate was washed with water (about 1 L) and then desorbed with a mixture of 75% ethanol and 25% water (about 1 L). Solvents were removed from the ethanolic eluate in vacuo (using a BÜCHI rotary evaporator) to obtain about 14.5 g of a brownish red solid (e.g., containing a mixture of GSE A, GSE B, and GSE C). The filter cake produced from the vacuum filtration was allowed to dry under ambient conditions to obtain about 204 g of a mixture containing GSE D and some insoluble solids.

In a separate experiment, a second aliquot (about 3.8 L) of the GSE retentate produce using the OMNIA microfilter was vacuum filtered through a BÜCHNER funnel with WHATMAN #1 filter paper. The solids and liquid retained by the vacuum filtration were centrifuged at about 8000 rpm, for about 5 min (using a SORVALL LYNX 6000 centrifuge, provided by THERMO SCIENTIFIC). The solids were washed with fresh water (about 100 mL) and mixed with high-proof ethanol (190 proof; about 1 L). The solids and ethanol were mixed using an immersion homogenizer (at about 2500 rpm) to facilitate dissolution of a portion of the solids in the ethanol. The mixture of solids, dissolved solids, and ethanol was vacuum filtered using a BÜCHNER funnel and WHATMAN #1 filter paper. The filtrate was rotary evaporated to dryness and dried further over phosphorus pentoxide (P₂O₅) to produce about 39.5 g of GSE D (a dark, crusty solid). Remaining solids were dried under ambient conditions to produce about 80.5 g of insoluble solids (e.g., solid material 544). Exemplary GSE compositions produced according to this method are described in Tables 8, 9, and 10.

### Example 6—Neuroprotective Effects of GSE A, B, C, and Combination Thereof

The following examples provides data evaluating the neuroprotective effects of various grape-derived extracts and fractions as described herein-specifically, GSE A (polymeric fraction), GSE B (oligomeric fraction), GSE C (monomeric fraction), and Grape MN Gold composite, which is comprises approximately 54% GSE A, 34% GSE B and 12% GSE C.

The oxytosis assay described in this example is a cell-based model that measures the ability of compounds to protect neuronal cells from oxidative stress-induced cell death. Oxytosis can be used interchangeably with ferroptosis. See Fischer et al. Redox Biol. 2019 February; 21:101089. In particular, the oxytosis assay, measures oxidative glutamate toxicity, is designed to evaluate the ability of compounds to protect neuronal cells from oxidative stress-induced programmed cell death, which is triggered by glutathione (GSH) depletion following glutamate exposure or exposure to other small molecules commonly used as inducers of oxidative stress and cell death in neuronal cell-based assays including erastin and RSL3. Erastin, similar to glutamate, blocks system xc-activity while RSL3 is an inhibitor of glutathione peroxidase 4 that is downstream of GSH loss and plays a key role in the cell death process in both oxytosis and ferroptosis. These pathways are mechanistically linked to aging and Alzheimer's disease, making the assay highly relevant for identifying neuroprotective agents with disease-modifying potential.

The ability of GSE A (polymeric fraction), GSE B (oligomeric fraction), GSE C (monomeric fraction), and the Grape MN Gold composite (approximately 54% GSE A, 34% GSE B and 12% GSE C) to protect neuronal cells from oxidative stress-induced cell death was evaluated and the results are summarized in Table 11 below (appropriate control samples were also included as shown). Experiments were designed and carried out consistent with those described in Fischer et al. Redox Biol. 2019 February; 21:101089 using HT22 mouse hippocampal nerve cells and BV-2 microglial cells.

TABLE 11
Oxytosis Assay Results using GSE A, GSE B, GSE C, and
Grape MN Gold.

| | | | | | Anti- |
|---|---|---|---|---|---|
| | | | | | inflam- |
| | | Oxytosis Inhibition - | | | matory |
| | | EC₅₀ (μg/mL) | | | EC₅₀ LPS |
| Sample | Description | glutamate | Erastin | RSL3 | (μg/ml) |
| Pure red grape | PRG + | no | no | no | no |
| treated | tannase | | | | |
| Pure red grape | PRG | no | no | no | no |
| control | | | | | |
| Pure purple | PPG + | no | no | no | no |
| grape treated | tannase | | | | |
| Pure purple | PPG | no | no | no | no |
| grape control | | | | | |
| GSE A | Polymeric | no | no | no | no |
| GSE B | Oligomeric | 47.5 | 46 | 61 | no |
| GSE C | Monomeric | 205 | 72.5 | 31 | 60 |
| Grape MN Gold | Composite | 26.5 | 22.5 | 28.5 | no |

The data presented in Table 11 demonstrates that GSE B, GSE C, and especially Grape MN Gold exhibit the ability to protect neuronal cells from oxidative stress-induced cell death. These readouts provide a comprehensive profile of each composition's neuroprotective and anti-inflammatory properties. The oligomeric and monomeric fractions, as well as the composite, may therefore be useful as dietary supplements, nutraceuticals, or pharmaceutical agents for improving cognition, slowing the progression of neurodegenerative diseases, or enhancing resilience to age-related neuronal loss. Additionally, the superior efficacy of the Grape MN Gold composite suggests that a combination of grape-derived polyphenols may provide broader neuroprotective benefits than isolated fractions.

Based on these results the grape seed extracts described herein and combinations thereof may also be useful for the treatment of metabolic syndrome, which is characterized by chronic low-grade inflammation, increased oxidative stress, and impaired glucose and lipid metabolism, all of which are risk factors for cognitive impairment and neurodegeneration. In particular, the ability of GSE B, GSE C, and Grape MN Gold to inhibit oxidative stress-induced neuronal death suggests that these extracts may help mitigate the neurocognitive complications associated with metabolic syndrome. By reducing oxidative damage and supporting neuronal survival, these grape-derived extracts can contribute to improved cognition. In summary, the data support the potential of GSE B, GSE C, and Grape MN Gold for improving cognition in a subject. Furthermore, the data suggests a synergistic effect when multiple grape seed extracts are combined using Grape MN Gold, resulting in greater efficacy than individual fractions alone.

It is understood that the present invention may take many forms and embodiments. Accordingly, several variations may be made in the foregoing without departing from the spirit or scope of the invention. Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

What is claimed is:

1. A grape seed extract composition for improving cognitive health comprising a first grape seed extract, a second grape seed extract, a third grape seed extract, or a combination thereof, wherein:
said first grape seed extract comprises a mean degree of polymerization from about 4.1 to about 5.3, and phenols in an amount from about 61 to about 70 g GAE/100 g, on a dry basis;
said second grape seed extract comprises a mean degree of polymerization from about 2.2 to about 2.7, and phenols in an amount from about 30 to about 60 g GAE/100 g, on a dry basis; and
said third grape seed extract comprises a mean degree of polymerization from about 1.2 to about 1.4, and phenols in an amount from about 80 to about 95 g GAE/100 g, on a dry basis.

2. The grape seed extract composition of claim 1, wherein the composition improves the cognition of a subject when delivered in an effective amount to said subject.

3. The grape seed extract of claim 1, wherein the composition comprises about 25-75 wt % of said first grape seed extract, about 15-45 wt % of said second grape seed extract, and about 2-20 wt % of said third grape seed extract, wherein the total percentage of the first, second, and third grape seed extracts equals 100%.

4. The grape seed extract of claim 3, wherein the composition comprises:
about 25-35 wt % of said first grape seed extract, about 40-45 wt % of said second grape seed extract, and about 20-30 wt % of said third grape seed extract;
about 35-45 wt % of said first grape seed extract, about 30-40 wt % of said second grape seed extract, and about 10-20 wt % of said third grape seed extract;
about 45-55 wt % of said first grape seed extract, about 25-35 wt % of said second grape seed extract, and about 5-15 wt % of said third grape seed extract;
about 55-65 wt % of said first grape seed extract, about 20-30 wt % of said second grape seed extract, and about 2-10 wt % of said third grape seed extract; or
about 65-75 wt % of said first grape seed extract, about 15-25 wt % of said second grape seed extract, and about 2-8 wt % of said third grape seed extract.

5. The grape seed extract of claim 3, wherein the composition comprises about 54 wt % of said first grape seed extract, about 34 wt % of said second grape seed extract, and about 12 wt % of said third grape seed extract.

6. The grape seed extract of claim 1, wherein the first grape seed extract further comprises at least one of:
a combination of catechin and epicatechin from about 0% to about 0.5%, by weight, on a dry basis;
an epicatechin gallate (ecg) terminal unit percentage from about 0 to about 16; or
an epicatechin gallate (ecg) extension unit percentage from about 5.6 to about 15.6.

7. The grape seed extract of claim 1, wherein the second grape seed extract further comprises at least one of:
gallic acid in an amount from about 0.3% to about 0.6%, by weight, on a dry basis;
a combination of catechin and epicatechin from about 0.3% to about 2.6%, by weight, on a dry basis;
an epicatechin gallate (ecg) terminal unit percentage from about 0 to about 14.4; or
an epicatechin gallate (ecg) extension unit percentage from about 3.7 to about 11.5.

8. The grape seed extract of claim 1, wherein the third grape seed extract further comprises at least one of:
gallic acid in an amount from about 0.4% to about 3.9%, by weight, on a dry basis;
a combination of catechin and epicatechin from about 20% to about 41%, by weight, on a dry basis;
an epicatechin gallate (ecg) terminal unit percentage from about 0.9 to about 9.1; or
an epicatechin gallate (ecg) extension unit percentage from about 1.4 to about 7.8.

9. A method of treating a subject, the method comprising administering to the subject a grape seed extract composition of claim 1, wherein the grape seed extract composition is administered in an amount effective to improve cognition of the subject.

10. The method of claim 9, wherein the grape seed extract composition is administered at about 100 mg/day to about 3000 mg/day, about 100 mg/day to about 2000 mg/day, about 100 mg/day to about 1000 mg/day, about 100 mg/day to about 500 mg/day, about 100 mg/day to about 300 mg/day, about 200 mg/day to about 3000 mg/day, about 400 mg/day to about 3000 mg/day, about 800 mg/day to about 3000 mg/day, about 1000 mg/day to about 3000 mg/day, about 1500 mg/day to about 3000 mg/day, about 2000 mg/day to about 3000 mg/day.

11. The method of claim 10, wherein the grape seed extract composition is administered at about 300 mg/day to about 1000 mg/day.

12. The method of claim 9, wherein the grape seed extract composition is administered in a capsule, a tablet, a liquid formulation, a lozenge, or a powder.

13. The method of claim 9, wherein the grape seed extract composition is administered about every 12 hours, about every 24 hours, about every 36 hours, about every 48 hours, about once weekly, about twice weekly, about every two weeks, about every three weeks, or about once every month.

14. The method of claim 9, wherein the subject is a human subject.

15. A grape seed extract composition produced by:
obtaining a grape seed extract source material;
filtering the grape seed extract source material to obtain a first retentate and a first permeate;
drying the first retentate to obtain a first grape seed extract comprising a first mean degree of polymerization;
filtering the first permeate to obtain a second retentate and a second permeate;
drying the second retentate to obtain a second grape seed extract comprising a second mean degree of polymerization less than the first mean degree of polymerization;
purifying the second permeate to obtain a purified liquid; and
drying the purified liquid to obtain a third grape seed extract comprising a third mean degree of polymerization less than the second mean degree of polymerization; and
combining the first, second, and third grape seed extracts;
wherein the grape seed extract composition comprises about 25-75 wt % of said first grape seed extract, about 15-45 wt % of said second grape seed extract, and about 2-20 wt % of said third grape seed extract, wherein the total percentage of the first, second, and third grape seed extracts equals 100%.

16. The grape seed extract composition of claim 15, wherein:
said first grape seed extract comprises a mean degree of polymerization from about 4.1 to about 5.3, and phenols in an amount from about 61 to about 70 g GAE/100 g, on a dry basis;
said second grape seed extract comprises a mean degree of polymerization from about 2.2 to about 2.7, and phenols in an amount from about 30 to about 60 g GAE/100 g, on a dry basis; or
said third grape seed extract comprises a mean degree of polymerization from about 1.2 to about 1.4, and phenols in an amount from about 80 to about 95 g GAE/100 g, on a dry basis.

17. The grape seed extract of claim 14, wherein the composition comprises:
about 25-35 wt % of said first grape seed extract in combination with about 40-45 wt % of said second grape seed extract, and about 20-30 wt % of said third grape seed extract;
about 35-45 wt % of said first grape seed extract, about 30-40 wt % of said second grape seed extract, and about 10-20 wt % of said third grape seed extract;
about 45-55 wt % of said first grape seed extract, about 25-35 wt % of said second grape seed extract, and about 5-15 wt % of said third grape seed extract;
about 55-65 wt % of said first grape seed extract, about 20-30 wt % of said second grape seed extract, and about 2-10 wt % of said third grape seed extract; or
about 65-75 wt % of said first grape seed extract, about 15-25 wt % of said second grape seed extract, and about 2-8 wt % of said third grape seed extract.

18. The grape seed extract of claim 17, wherein the composition comprises about 54 wt % of said first grape seed extract, about 34 wt % of said second grape seed extract, and about 12 wt % of said third grape seed extract.

19. The grape seed extract composition of claim 15, wherein the composition improves the cognition of a subject when delivered in an effective amount to said subject.

20. The grape seed extract composition of claim 19, wherein the subject exhibits improvements on a standardized cognition test.

21. The grape seed extract composition of claim 20, wherein the standardized cognition test is selected from the group consisting of Montreal Cognitive Assessment, Mini-Mental State Examination, Mini-Cog, and General Practitioner Assessment of Cognition.
